(19) United States
(12) Patent Application Publication
(10) Pub. No.: US 2020/0368394 A1
(43) Pub. Date: Nov. 26, 2020

(54) COMPOSITIONS AND METHODS FOR RESTORING OR PREVENTING LOSS OF VISION CAUSED BY DISEASE OR TRAUMATIC INJURY

(71) Applicant: BIOTIME, INC., Alameda, CA (US)

(72) Inventors: Igor NASONKIN, Alameda, CA (US); Ratnesh SINGH, San Ramon, CA (US); Oscar CUZZANI, Walnut Creek, CA (US); Michael ONORATO, San Francisco, CA (US); Francois BINETTE, Napa, CA (US)

(73) Assignee: Lineage Therapeutics, Inc., Carlsbad, CA (US)

(21) Appl. No.: 16/635,473

(22) PCT Filed: Jul. 31, 2018
(86) PCT No.: PCT/US2018/044720

§ 371 Date: Jan. 30, 2020

Related U.S. Application Data
(60) Provisional application No. 62/665,483, filed on May 1, 2018.
(60) Provisional application No. 62/646,354, filed on Mar. 21, 2018.
(60) Provisional application No. 62/593,228, filed on Nov. 30, 2017.
(60) Provisional application No. 62/577,154, filed on Oct. 25, 2017.
(60) Provisional application No. 62/539,542, filed on Jul. 31, 2017.

(51) Int. Cl. A61L 27/36; A61K 35/545; A61K 31/137; A61F 2/14; A61L 27/54

(52) U.S. Cl. CPC A61L 27/3666; A61K 35/545; A61K 31/137; A61L 2430/16; A61F 2/148; A61L 27/54; A61L 27/3604

(57) ABSTRACT

Bioprosthetic retinal grafts (or devices) comprising stem cell derived tissues and/or cells may be used to slow the progression of retinal degenerative disease, slow the progression of retinal degenerative disease after traumatic injury, slow the progression of age related macular degeneration (AMD), prevent retinal degenerative disease, prevent retinal degenerative disease after traumatic injury, prevent AMD, restore retinal pigment epithelium (RPE), photoreceptor cells (PRCs) and retinal ganglion cells (RGCs) lost from disease, injury or genetic abnormalities, increasing RPE, PRCs and RCGs, treat RPE, PRCs and RCG defects in a subject, or for other purposes. Bioprosthetic retinal grafts may comprise a bioprosthetic carrier or scaffold suitable for implantation into the ocular space of a subject's eye, to form a bioprosthetic retinal patch. In certain embodiments, the bioprosthetic retinal patch may comprise multiple pieces of stem cell derived tissues or cells on a carrier or scaffold, which may be used to treat large areas of retinal degeneration or damage, or for other purposes.

### CROSS-REFERENCE TO RELATED APPLICATIONS

This application claims priority to, and the benefit of, U.S. provisional patent application Ser. No. 62/539,542 filed on Jul. 31, 2017, U.S. provisional patent application Ser. No. 62/577,154 filed on Oct. 25, 2017, U.S. provisional patent application Ser. No. 62/593,228 filed on Nov. 30, 2017, U.S. provisional patent application Ser. No. 62/646,354 filed on Mar. 21, 2018, and U.S. provisional patent application Ser. No. 62/665,483 filed on May 1, 2018, the entire content of each of these documents being incorporated herein by reference in their entirety.

### BACKGROUND

Retinal degenerative (RD) diseases, which ultimately lead to the degeneration of photoreceptors (PRs), are the third leading cause of blindness worldwide. Genetic conditions, age and trauma (military and civilian) are leading causes of vision loss associated with retinal degenerations. Once photoreceptors are degenerated, there is no current technology to restore retina and bring vision back.

Age-Related Macular Degeneration (AMD) is a leading cause of RD in people over 55 years old in developed countries. About 15 million people in the US are currently affected by AMD, which accounts for about 50% of all vision loss in the US and Canada. Retinitis pigmentosa (RP) is the most frequent cause of inherited visual impairment, with a prevalence of 1:4000, and is estimated to affect 50,000 to 100,000 people in the United States and approximately 1.5 million people worldwide. Other retinal diseases which cause severe vision loss include Leber's Congenital Amaurosis (LCA), a rare genetic disorder in which retinal dysfunction causes vision loss, often from birth. The extent of vision loss varies from patient to patient but can be quite severe (with little to no light perception).

As personal ballistic protection of the head and torso offers increased combat protection, there are increasing numbers of soldiers surviving injuries to less protected areas of the body such as the face and eyes. Ocular injury resulting from blast exposure is the fourth most common injury sustained in military combat. Ocular injury often leads to blindness, causing devastating loss of quality of life and independence. Although penetrating injuries often result in severe tissue damage or tissue loss, non-penetrating or closed globe injuries can similarly result in disruption of the highly-ordered tissue architecture in the eye, causing retinal detachment, photoreceptor cell death, and optic nerve damage, leading to irreversible vision loss. Closed globe injuries often present an injury pattern wherein ocular structures remain largely intact yet require intervention to prevent degeneration of the retina and optic nerve resulting in devastating vision loss.

A recently developed strategy for restoring vision in RD patients is implantation of electronic neuroprosthetic chips, which introduce light-capturing sensors into the subretinal space to transmit visual signals electrically to the remaining neurons in a patient's retina. One problem with this approach is the gradual separation of electronic and biological parts due to ongoing retinal degeneration and remodeling, thinning of retina, and gliosis, further reducing chip-to-retina interaction, which is critical for transducing electrical signals. Additional issues are caused by limited stability of an electronic device in biological tissue, where metals and wiring used in the chips undergo oxidation, caused by biological fluids.

Retinal tissue transplantation using human fetal retina has also been demonstrated to restore visual perception in blind animals and also improve vision in patients with retinal degeneration. Though the approach is promising and produces a new layer of healthy human retina in a patient's subretinal space, the use of fetal tissue as a treatment option is hindered by ethical considerations and a scarce and unpredictable supply of fetal tissue. In addition, the success of the vision restoration procedure depends on selecting human fetal retina of a specific developmental age (8-17 weeks) and precisely placing it into patient's subretinal space. Adult retina on its own is generally not suitable on for this application, because it rapidly dies after transplantation.

Among all stem cell replacement therapies, retinal stem cell therapy stands out because it is one of the most urgent unmet needs. The eye is a small, encapsulated organ, with immune privilege. The ocular space is accessible for transplantation and the retina can be visualized using noninvasive methods. But repairing the neural retina by functional cell replacement is a complex task. For best results, the new cells must migrate to specific locations in the retinal layers and re-establish specific synaptic connectivity with the host. Synaptic remodeling of neural circuits during advanced RD further complicates this task.

Thus, there is a need for robust and feasible treatments for vision restoration technologies focused on restoration and protection of structure and function following retinal injury or disease, whereby retinal damage can be severe, affect a large portion of the retina or cause ongoing degeneration over time.

The present disclosure addresses these and other shortcomings in the field of regenerative medicine and cell therapy.

### BRIEF SUMMARY

In one aspect, a method is provided for one or more of, treating retinal damage, slowing the progression of retinal damage, preventing retinal damage, replacing retinal tissue and restoring damaged retinal tissue, the method comprising: administering a hESC-derived retinal tissue graft to a subject.

In another aspect, a method is provided for one or more of, slowing the progression of retinal degenerative disease, slowing the progression of retinal degenerative disease after traumatic injury, slowing the progression of age related macular degeneration (AMD), slowing the progression of genetic retinal diseases, stabilizing retinal disease, preventing retinal degenerative disease, preventing retinal degenerative disease after traumatic injury, improving vision or visual perception, preventing AMD, restoring retinal pigment epithelium (RPE), photoreceptor cells (PRCs) and retinal ganglion cells (RGCs) lost from disease, injury or genetic abnormalities, increasing RPE, PRCs and RCGs or treating RPE, PRCs and RCG defects, the method comprising: administering a hESC-derived retinal tissue graft to a subject.

In another aspect, retinal damage is caused by one or more of, blast exposure, genetic disorder, retinal disease, and retinal injury. In another aspect, retinal disease comprises a retinal degenerative disease. In another aspect, retinal damage is caused by one or more of, Age-Related Macular Degeneration (AMD), retinitis pigmentosa (RP), and Leber's Congenital Amaurosis (LCA).

In one embodiment, methods described use hESC derived retinal tissue comprises retinal pigmented epithelial (RPE) cells, retinal ganglion cells (RGCs), and photoreceptor (PR) cells. In another embodiment, the RPE, RGC and PR cells are configured such that there is a central layer of retinal pigmented epithelial (RPE) cells, and, moving radially outward from the RPE cell layer, a layer of retinal ganglion cells (RGCs), a layer of second-order retinal neurons (corresponding to the inner nuclear layer of the mature retina), a layer of photoreceptor (PR) cells, and an outer layer of RPE cells. In another embodiment, each of the layers comprise differentiated cells characteristic of the cells within the corresponding layer of human retinal tissue. In another embodiment, each of the layers comprise progenitor cells and wherein some or all or the progenitor cells differentiate into mature cells of the corresponding layer of human retinal tissue after administration.

In another embodiment, the layers comprise substantially fully differentiated cells. In yet another embodiment, the hESC-derived retinal tissue further comprises a biocompatible scaffold to form a bioprosthetic retinal patch. In other embodiments, the bioprosthetic retinal graft comprises between about 10,000 and 100,000 photoreceptor cells. In other embodiments, several pieces of the hESC-derived retinal tissue are affixed to the biocompatible scaffold, such that a large bioprosthetic patch is formed. In other embodiments, the hESC-derived retinal tissue graft or dissociated cells of the hESC derived retinal tissue graft are capable of delivering to a subject one or more of, neurotrophic factors, neurotrophic exosomes and mitogens. In yet other embodiments, the neurotrophic factors and mitogens comprise one or more of, brain-derived neurotrophic factor (BDNF), glial-derived neurotrophic factor (GDNF), neurotrophin-34 (NT34), neurotrophin 4/5, Nerve Growth Factor-beta (βNGF), proNGF, PEDF, CNTF, pro-survival mitogen basic fibroblast growth factor (bFGF=FGF-2) and pro-survival members of the WNT family.

In other aspects, administration of the hESC-derived retinal tissue graft results in preservation of retinal layer thickness for between about 1 to about 3 months where administered. In yet other aspects, administration further comprises administration of immunosuppressive drugs. In other aspects, administration comprises use of epinephrine before, during and/or after administering the retinal graft.

In yet other aspects, the immunosuppressive drugs are administered before, during and/or after the administration.

In other embodiments, the methods further comprises modulating the ocular pressure. In other aspects, the modulating the ocular pressure is before, during and/or after the administration of the retinal tissue.

In certain embodiments, the tissue is administered with an ocular grafting tool.

In other embodiments, the hESC-derived retinal tissue is administered subretinally or epiretinally.

In other embodiments, administration of the hESC-derived retinal tissue graft results in tumor-free integration of the hESC-derived retinal tissue and retinal tissue of the subject.

In other embodiments, integration of retinal graft occurs between about 2 to 10 weeks after administration. In other embodiments, integration comprises structural integration. In other embodiments, integration comprises functional integration and occurs between about 1 to 6 months after administration. In other embodiments, administering does not cause retinal inflammation.

In other embodiments, after administering, the retinal tissue develops lamination.

In other embodiments, after administering, the retinal tissue neurons show signs of Na⁺, K⁺ and/or Ca⁺ currents.

In other embodiments, methods further comprise, demonstrating connectivity between the retinal tissue and existing tissue. In other embodiments, the connection is demonstrated by one or more of: WGA-HRP trans-synaptic tracer, histology, IHC or electrophysiology.

In other embodiments, methods further comprise, measuring a level of functional recovery.

In other embodiments, a level of functional recovery comprises a gain in the electrophysiological responses that is at least 10% of a baseline.

In other embodiments, a retinal tissue graft for transplantation into an eye of a subject, comprising: retinal pigmented epithelial (RPE) cells, retinal ganglion cells (RGCs), second-order retinal neurons, and photoreceptor (PR) cells, wherein the RPE, RGC and PR cells are configured to form a central core is presented.

In other embodiments, there are from between about 1,000 and 250,000 photoreceptors.

In other embodiments, the second-order retinal neurons correspond to the inner nuclear layer of the mature retina.

In other embodiments, the cells are arranged such that moving radially outward from the core, the retinal tissue comprises a layer of retinal ganglion cells (RGCs), a layer of second-order retinal neurons, a layer of photoreceptor (PR) cells, and an outer layer of RPE cells. In other embodiments, the graft comprises from between 1,000 to about 250,000 cells.

In other embodiments, the graft is transplanted into the subretinal space or epiretinal space.

In other embodiments, the graft is transplanted into the subretinal space or epiretinal space near the macula. In other embodiments, an increase in synaptogenesis coincides with increase in electric activity.

In other embodiments, after transplantation neurons connect the graft to existing tissue.

In other embodiments, the neurons are CALB2-positive. In other embodiments, connectivity is demonstrated by WGA-HRP trans-synaptic tracer. In other embodiments, after transplantation axons connect the graft to existing tissue. In other embodiments, the axons are CALB2-positive.

In other embodiments, after transplantation, cells of the graft mature toward RGCs.

In other embodiments, after transplantation the graft forms synapses with existing neurons.

In other embodiments, after transplantation the graft and existing tissue form connections.

In other embodiments, the connections form within one day to about 5 weeks after transplantation.

In other embodiments, after transplantation the graft forms axons which cross the existing tissue ONL.

In other embodiments, the graft produces paracrine factors.

In other embodiments, the paracrine factors are produced prior and/or after to administration.

In other embodiments, the graft produces neurotrophic factors.

In other embodiments, the graft produces neurotrophic factors prior to or after administration.

In other embodiments, the neurotrophic factors comprise one or more of, BDNS, GDNF, bNGF, NT4, bFGF, NT34, NT4/5, CNTF, PEDF, serpins, or WNT family members.

In other embodiments, after transplantation, the level of functional recovery is measured as a gain in the electrophysiological responses.

In other embodiments, the level of functional recovery is measured as a gain in the electrophysiological responses to at least 10% of a baseline.

In other embodiments, after transplantation, axons of the graft penetrate and integrate into existing tissue.

In other embodiments, the tissue is derived from human pluripotent stem cells.

In other embodiments, the graft is useful for slowing the progression of retinal degenerative disease, slowing the progression of retinal degenerative disease after traumatic injury, slowing the progression of age related macular degeneration (AMD), slowing the progression of genetic retinal diseases, stabilizing retinal disease, preventing retinal degenerative disease, preventing retinal degenerative disease after traumatic injury, improving vision or visual perception, preventing AMD, restoring retinal pigment epithelium (RPE), photoreceptor cells (PRCs) and retinal ganglion cells (RGCs) lost from disease, injury or genetic abnormalities, increasing RPE, PRCs and RCGs or treating RPE, PRCs and RCG defects, in a subject.

In other embodiments, the graft is capable of tumor-free survival for at least about 6 to 24 months, with lamination and development of PR and RPE layers, including elongating PR outer segments, synaptogenesis, electrophysiological activity and connectivity with recipient retinal cells after implantation into a recipient's ocular space.

In other embodiments, the graft is capable of extending and integrating axons into a recipient's outer nuclear layer (ONL), into the inner nuclear layer (INL) and into the ganglion cell layer (GCL) after 5 weeks after the graft is implanted into the ocular space of the recipient's eye.

Methods are provided herein for restoring vision loss or slowing the progression of vision loss, by administering a retinal patch. In one aspect, a vison restoration or improvement product is provided which can be injected or introduced into the epiretinal or subretinal space of a patient's eye.

In another aspect, a method of correcting loss of vision in a subject with a damaged retina is provided, the method comprising restoring retinal tissue to the damaged area. In yet another aspect, a method of correcting loss of vision in a subject is provided, wherein damaged retinal tissue is restored by administering a biological retinal patch to the damaged area. In another aspect, a method of correcting loss of vision in a subject with a damaged retina by administering a biological retinal patch is provided, wherein the biological retinal patch comprises: engineered retinal tissue; electrospun biopolymer scaffold; and adhesive; wherein the retinal tissue is fastened to the biopolymer by the adhesive.

Further aspects and embodiments are described infra.

### BRIEF DESCRIPTION OF THE DRAWINGS

The technology described herein will be more fully understood by reference to the following drawings which are for illustrative purposes only:

FIG. 1A shows an illustration of a subretinal graft, according to certain embodiments of the present disclosure.

FIG. 1B shows an illustration of a bioprosthetic retinal patch comprising, hPSC derived retinal tissue (organoids) and a bioprosthetic scaffold support, according to certain embodiments.

FIG. 1C shows an illustration of a bioprosthetic retinal patch comprising, many hPSC derived retinal tissue pieces and a bioprosthetic scaffold support, according to certain embodiments.

FIG. 1D shows an illustration of a bioprosthetic retinal patch comprising, hPSC derived retinal tissue (organoids), a bioprosthetic scaffold support, and an RPE component, according to certain embodiments.

FIG. 1E shows an illustration of a of a bioprosthetic retinal patch comprising, hPSC derived retinal tissue, a bioprosthetic scaffold support, and a photosensitive diode (photo diode) component, according to certain embodiments.

FIG. 2 shows a chart describing the Birmingham Eye Trauma Terminology System (BETTS).

FIG. 3A shows images of hPSC derived retinal tissue stained with antibodies specific for the Calretinin marker, CALB2, which is expressed in neurons, including retina.

FIG. 3B shows images of hPSC derived retinal tissue stained with antibodies specific for the retinal cytoplasmic marker, Recoverin (RCVRN).

FIG. 3C shows grafts of FACS-sorted PR cells from retinal organoids (retinal tissue bioprosthetic grafts) as compared to human fetal retina.

FIG. 4A shows an ICH image of retinal integration and maturation of hESC derived retinal progenitor cells (hESC-RPCs) transplanted into the epiretinal space of a mouse model. As shown, most of the human progenitor cells are negative for the early neuronal marker, Tuj1, and can be seen migrating and integrating into the host's retinal ganglion cell (RGC) layer or inner nuclear layer (INL).

FIG. 4B shows an ICH image of implanted hESC derived retinal progenitor cells migrating over a large area of the host's subretinal area.

FIG. 4C shows an ICH image of cells from implanted epiretinal hESC-RPCs integrating into the host's retinal ganglion cell (RGC) layer, inner plexiform layer, and inner nuclear layer (INL).

FIG. 5A shows an image of the retinal tissue bioprosthetic graft transplantation.

FIG. 5B shows an ICH image of stained epiretinal grafts of hESC-RPCs in rabbit eyes. Part of the human retinal organoid is stained with the human nuclear marker, HNu, and shows human retinal progenitor cells from human retinal organoids grafted into the epiretinal space of a rabbit eye. The sample was also counterstained with DAPI.

FIG. 5C shows an ICH image of stained epiretinal grafts of hESC-RPCs in rabbit eyes. Part of the human retinal organoid is stained with the human nuclear marker, HNu, and shows human retinal progenitor cells from human retinal organoids grafted in the epiretinal space of a rabbit eye.

FIG. 5D shows an ICH image of a human retinal organoid in a large animal model (rabbit) and demonstrated that retinal organoids described herein can be delivered into the ocular space of a rabbits (a large eye animal model) using a glass canula through an incision in the pars plana without damage to the eye. The eye was successfully preserved and stained, showing the location of the human retinal cells.

FIG. 6 shows a schematic diagram and corresponding image of the shock tube, according to certain embodiments.

FIG. 7A shows the risk curve for the retina. The probabilities for achieving an injury with a given CIS at a specific blast intensity (expressed as the specific impulse in kPa-ms) are shown by the curves (red=CIS 1; green=CIS 2; CIS 3; black=CIS 4).

FIG. 7B shows the risk curve for the optic nerve. The probabilities for achieving an injury with a given CIS at a specific blast intensity (expressed as the specific impulse in kPa-ms) are shown by the curves (red=CIS 1; green=CIS 2; CIS 3; black=CIS 4).

FIG. 8 is an OCT image of hESC derived retinal tissue graft in the subretinal space of a large eye animal model (wild type cat) after transplantation.

FIG. 9 is an image of immunostaining of the hESC derived retina with HNu antibody in the cat eye after transplantation which shows the presence of the retinal graft in the correct location.

FIG. 10A shows an image of hESC-3D derived retinal tissue (retinal organoids) dissected from a dish before transplantation.

FIG. 10B shows an image of the dissected hESC-3D derived retinal organoids growing on a dish before transplantation.

FIG. 10C shows an additional image of hESC-3D derived retinal organoids growing on a dish.

FIG. 10D shows an IHC image of a hESC-3D derived retinal tissue bioprosthetic graft in blind immunodeficient rat eye, demonstrating layering and lamination of the graft after administration.

FIG. 10E shows an IHC image of a hESC-3D derived retinal tissue bioprosthetic graft, demonstrating layering and lamination of the graft.

FIG. 10F shows an ICH image of a hESC-3D derived retinal tissue bioprosthetic graft implanted into blind immunodeficient rat eye with outer segment-like protrusions in the outer layer, immediately next to rat RPE.

FIG. 11 shows ICH images demonstrating maintained retinal tissue viability after an overnight shipment in Hib-E at 4° C. The arrows highlight the viable human implanted cells.

FIG. 12A through FIG. 12C show images of a surgical team transplanting hESC-3D retinal tissue in subretinal space of a wild type cat.

FIG. 12D shows an image of the equipment for modulating ocular pressure and, RetCam equipment for imaging the grafts.

FIG. 12E shows two ports inserted in a cat eye for intraocular surgery.

FIG. 12F shows retinal detachment (a bleb), for grafting hESC-3D retinal tissue bioprosthetic grafts into the subretinal space.

FIG. 12G shows a cannula for injecting hESC-3D retinal tissue.

FIG. 12h shows hESC-3D retinal tissue in the subretinal space of a wild type cat, imaged with a RetCam.

FIG. 12I shows the location of an OCT image of hESC-3D retinal tissue placed in the subretinal space of a wild type cat, 5 weeks after grafting.

FIG. 12J shows a cross-sectional OCT image of hESC-3D retinal tissue placed in the subretinal space of a wild type cat, 5 weeks after grafting.

FIG. 12K shows a 3D reconstruction of an OCT image to estimate the total size of the graft.

FIG. 13A shows a PFA-fixed, cryoprotected, OCT-saturated cat eye with subretinal graft, prepared for sectioning.

FIG. 13B shows a cross-section of a cat eye frozen in OCT.

FIG. 13C shows 16-μm-thick sections of a cat eye in OCT, which shows the graft as a bulge in the central retina.

FIG. 13D shows a magnified image of the area of a frozen section showing preservation of hESC-3D retinal tissue grafts.

FIG. 13E shows IHC images of a section of cat retina with hESC-3D retinal tissue graft, 5 weeks after grafting into the subretinal space. The graft shows the presence of many CALB2 (Calretinin)-positive neurons and the arrows point to CALB2[+] axons connecting human graft and cat's ONL.

FIG. 13E through FIG. 13G show images of the hESC-3D retinal tissue graft in a cat's subretinal space, stained with HNu, Ku80 and SC121 human (but not cat)-specific antibodies, respectively. These results demonstrate that human tissue was in fact grafted into the correct location of the cat's subretinal space.

FIG. 13H shows images of staining with BRN3A (marker of RGCs) and Human nuclei marker. The asterisks show the area with the markers in the main image, which are enlarged in the insets. These results indicate that some cells within the graft are undergoing maturation towards RGCs.

FIG. 13I through FIG. 13M show images of staining with antibodies specific to human (but not cat)-synaptophysin (hSYP) and axonal marker NFL (specific to both cat and human neurons) and shows the presence of puncta-like staining (arrows) which indicates potential synapses formed by human neurons, which are integrating into cat neurons.

FIG. 14A and FIG. 14B show images of human (but not cat)-specific synaptophysin antibody hSYP (Red) and Calretinin (Green), which stains both cat and human neurons.

FIG. 14C and FIG. 14D show images of lower magnification images, providing an overview on the large piece of cat retina with the hESC-3D retinal tissue graft.

FIG. 15A through FIG. 15C show images of Calretinin[+] axons (arrows) connecting the cat INL and the Calretinin[+] human cells in the graft.

FIG. 15D and FIG. 15E show images of Calretinin[+] neurons in the graft, which look mature and Calretinin[+] axons which were found throughout the grafts.

FIG. 16A through FIG. 16C show images of staining of the edge of the hESC-3D retinal tissue graft in the cat subretinal space. SC121 human cytoplasm-specific antibody (Red) and Ku80 human nuclei specific antibody (Green) stain human retinal graft but not cat retina. It can be seen from these images that there is graft to host connectivity.

FIG. 16D and FIG. 16E shows images of the axons from hESC-3D retinal tissue graft wrap around (arrows) the cat PRs in the layer immediately next to the graft, while some SC121+ human axons can be seen crossing cat's ONL (arrows).

FIG. 17 shows a RetCam image of an implanted retinal organoids in a cat—imaged immediately post grafting into subretinal space.

FIG. 18A and FIG. 18B show illustrations comparing human and cat eye structure.

FIG. 19 shows an example of a timeline for the differentiation of retinal organoids, according to certain embodiments.

FIG. 20A through FIG. 20I show images of retinal progenitor markers and early photoreceptor markers in hESC-derived retinal tissue.

FIG. 21 shows an image of the transplantation of a hESC derived retinal tissue bioprosthetic graft into the subretinal space of a wild type cat eye following a pars plana vitrectomy using a glass cannula.

FIG. 22 shows an image of the subretinal bleb into which a hESC derived retinal tissue bioprosthetic graft is transplanted.

FIG. 23 shows color fundus and OCT images taken at three weeks after grafting of a hESC derived retinal tissue bioprosthetic graft.

FIG. 24 shows an image of a retinal section from a cat retina in Group 1 (+Prednisone, −Cyclosporine A), stained using antibodies specific for microglia and macrophages.

FIG. 25 shows an image of a retinal section taken from a cat retina in Group 2 (+Prednisone, +Cyclosporine A), also stained using antibodies specific for microglia and macrophages.

FIG. 26 shows a graph comparing the number of cells that are positive for microglia and macrophage cell markers in cat retinal sections for Group 1 (+Prednisone, −Cyclosporine A) and Group 2 (+Prednisone, +Cyclosporine A).

FIG. 27A shows an image of a cat retinal section from Group 2 (+Prednisone, +Cyclosporine A) stained using antibodies specific for the photoreceptor marker, CRX.

FIG. 27B shows an image of a cat retinal section from Group 2 (+Prednisone, +Cyclosporine A) stained using human-specific antibodies, HNu.

FIG. 27C shows an image of a cat retinal section from Group 2 (+Prednisone, +Cyclosporine A) stained using antibodies to both CRX and HNu.

FIG. 28A shows an image of a section of cat retina from Group 2 (+Prednisone, +Cyclosporine A) stained using antibodies specific for the retinal ganglion cell (RGC) marker, BRN3A.

FIG. 28B shows an image of a section of cat retina from Group 2 stained with both BRN3A and the human specific marker, KU80.

FIG. 28C shows an image of a section of cat retina from Group 2 stained with BRN3A, the human specific marker, KU80 and DAPI.

FIG. 29A shows an image of a cat retinal section stained using antibodies specific for the Calretinin marker, CALB2, which is expressed in neurons, including retina.

FIG. 29B shows an image of IHC staining for the marker, SC121. Antibodies to the SC121 are specific for human cell cytoplasm.

FIG. 29C shows an image of a cat retinal section stained using antibodies specific for the markers, CALB2, SC121 and DAPI.

FIG. 30A shows an ICH image of the axons of the retinal graft (stained using antibodies specific for the CALB2 marker) extending towards the cat retina.

FIG. 30B shows an ICH image of the retinal graft stained with antibodies specific for the human cell marker, HNu and CALB2, thereby delineating the graft from the cat retina.

FIG. 30C shows an ICH image of GABA positive staining of the graft axons, indicating that the axons from the implanted tissue integrating into the recipient retina are differentiating towards a neuronal fate.

FIG. 31A through FIG. 31G show OCT images of human ESC-derived retinal organoids in the subretinal and epiretinal space of CRX-mutant cats with retinal degeneration (RD).

FIG. 32 shows an ICH image of a bioprosthetic retinal graft comprising hESC derived retinal tissue positive for the expression of BDNF 5 weeks after administration of the graft into the subretinal space of a wild type cat eye.

### DETAILED DESCRIPTION

Bioprosthetic retinal grafts (or devices) described herein may be used to treat retinal degenerative diseases and disorders. For example, bioprosthetic retinal grafts may comprise stem cell derived tissues or cells. In some embodiments, the bioprosthetic retinal grafts may also comprise a carrier or scaffold, suitable for implantation into the ocular space of a subject's eye, to form a bioprosthetic retinal patch. In certain embodiments, the bioprosthetic retinal patch may comprise multiple pieces of stem cell derived tissues or cells on a carrier or scaffold, which may be used to treat large areas of retinal degeneration or damage.

The present disclosure relates to cell and/or tissue compositions and methods of formulating cell and/or tissue compositions suitable for therapeutic use in slowing the progression of retinal degenerative disease, slowing the progression of retinal degenerative disease after traumatic injury, slowing the progression of age related macular degeneration (AMD), preventing retinal degenerative disease, preventing retinal degenerative disease after traumatic injury, preventing AMD, restoring retinal pigment epithelium (RPE), photoreceptor cells (PRCs) and retinal ganglion cells (RGCs) lost from disease, injury or genetic abnormalities, increasing RPE, PRCs and RCGs or treating RPE, PRCs and RCG defects in a subject.

The term “subject,” as used herein includes, but is not limited to, humans, non-human primates and non-human vertebrates such as wild, domestic and farm animals including any mammal, such as cats, dogs, cows, sheep, pigs, horses, rabbits, rodents such as mice and rats. In some embodiments, the term “subject,” refers to a male. In some embodiments, the term “subject,” refers to a female.

The terms “treatment,” “treat” “treated,” or “treating,” as used herein, can refer to both therapeutic treatment or prophylactic or preventative measures, wherein the object is to prevent or slow down (lessen) an undesired physiological condition, symptom, disorder or disease, or to obtain beneficial or desired clinical results. In some embodiments, the term may refer to both treating and preventing. For the purposes of this disclosure, beneficial or desired clinical results may include, but are not limited to one or more of the following: alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response. Treatment also includes prolonging survival as compared to expected survival if not receiving treatment.

### Retinal Implants

Aspects of the present disclosure provide compositions and methods for treating, restoring and/or improving loss of vision caused by traumatic injury or disease in a subject by restoring retinal tissue to the damaged area. In certain embodiments, the disclosure provides methods for restoring loss of vision in a subject using for example, biocompatible, resorbable matrices, scaffolds and/or carriers to deliver engineered retinal tissue to the affected area. For retinal tissue engineering and delivery applications, wherein there is a large area of damaged tissue, it is beneficial to create a biocompatible scaffold in which to attach a large amount of engineered retinal tissue for controlled placement within a subject's eye.

In one aspect, a transplantable biological retinal patch or biological retinal prosthetic device derived from human pluripotent stem cells (hPSC), human embryonic stem cells (hESC) and/or tissue, and/or human fetal retinal tissue or adult retinal tissue, useful for restoring vision after extensive closed globe and retinal injury, slowing the progression of retinal degenerative disease, slowing the progression of retinal degenerative disease after traumatic injury, slowing the progression of age related macular degeneration (AMD), preventing retinal degenerative disease, preventing retinal degenerative disease after traumatic injury, preventing AMD, restoring retinal pigment epithelium (RPE), photoreceptor cells (PRCs) and retinal ganglion cells (RGCs) lost from disease, injury or genetic abnormalities, increasing RPE, PRCs and RCGs or treating RPE, PRCs and RCG defects in a subject is presented.

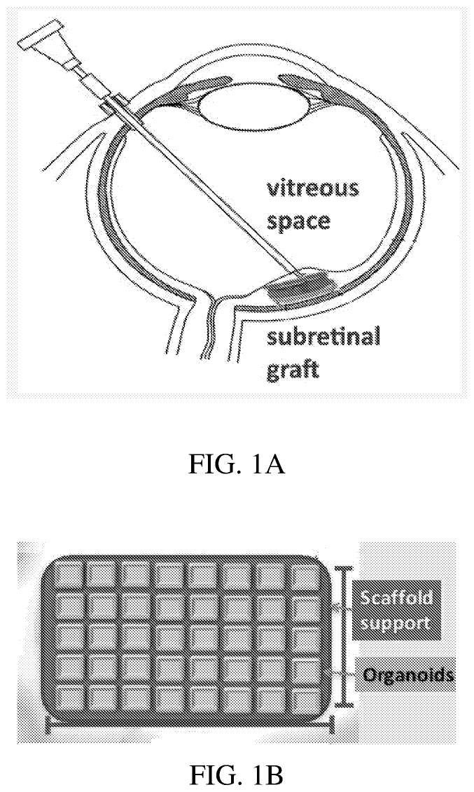

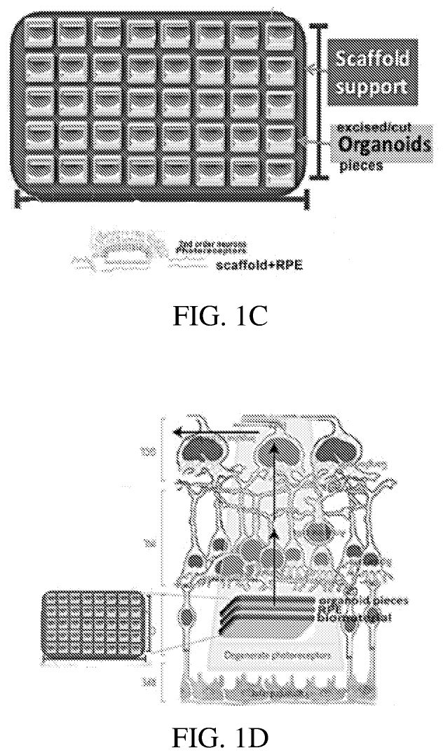

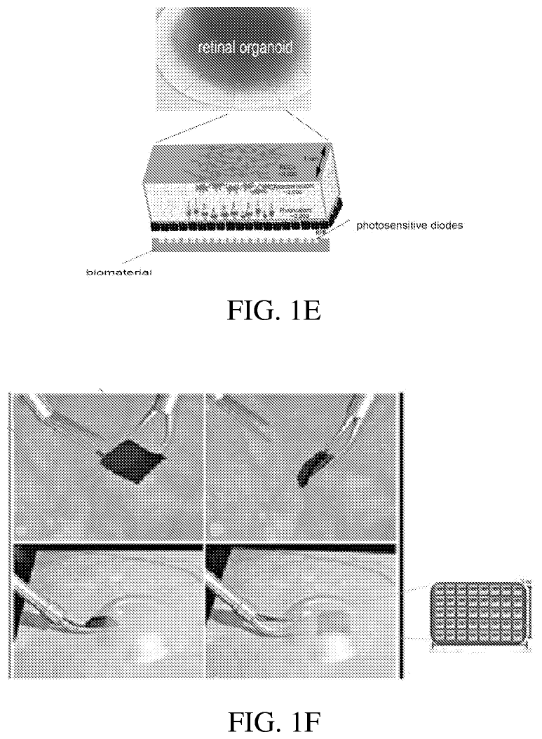

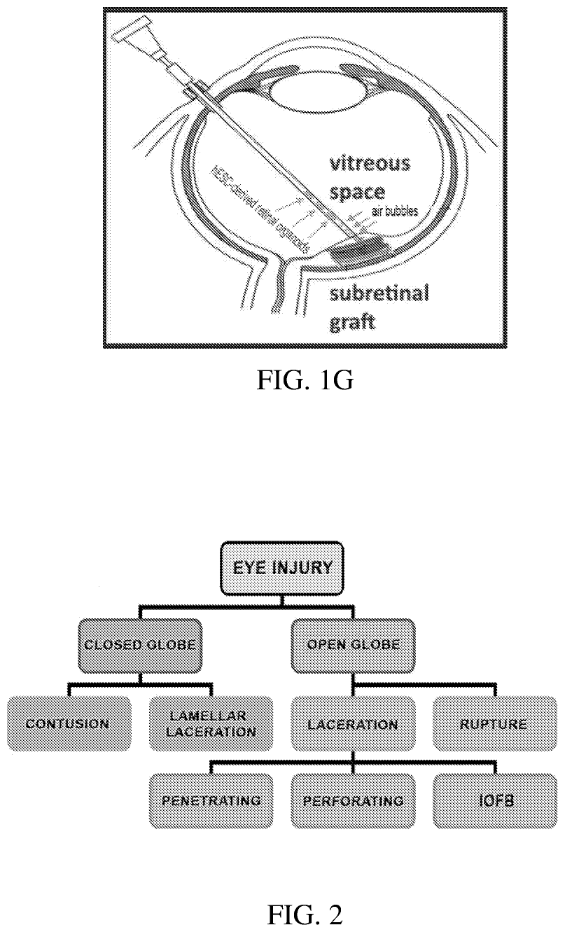

FIG. 1A shows an illustration of a subretinal graft being implanted into the subretinal space of a subject's eye, according to certain embodiments of the present disclosure. FIG. 1B shows an illustration of a bioprosthetic retinal patch, comprising hPSC derived tissue (organoids) and a bioprosthetic scaffold support.

In one aspect, human pluripotent (or embryonic) stem cell-derived tissue (hPSC derived retinal tissue or hPSC-3D retinal tissue) can be used for transplantation into a subject's ocular subretinal or epiretinal space. hPSC-3D retinal tissue represents a significant advancement in vision restoration therapeutics, as retinal tissue produced from hESCs maintain an innate ability to complete differentiation following transplantation and to reestablish synaptic connectivity with a recipient's retina. A small slice of hESC-3D retinal tissue can comprise from between about 1,000 to 2,000 photoreceptors or 2,000 to 3,000, or 1,000 to 5,000, 3,000 to 10,000, or 5,000 to 100,000, or 50,000 to 500,000 or 100,000 to 1,000,000 or more photoreceptors, the critical light sensing cells. Placing many individual pieces of hESC-3D retinal tissue on a single patch of very thin biomaterial can produce a large and flexible (yet, transplantable) biological retinal tissue bioprosthetic patch for vision improvement. This retinal tissue vision correction product can reduce surgical mistakes, as grafts and patched described herein allow for precise and controlled placement of the retinal tissue graft.

In certain embodiments, three-dimensional in vitro engineered retinal tissue, in the approximate shape of a flattened cylinder (or disc) contains a central core of retinal pigmented epithelial (RPE) cells, and, moving radially outward from the RPE cell core, a layer of retinal ganglion cells (RGCs), a layer of second-order retinal neurons (corresponding to the inner nuclear layer of the mature retina), a layer of photoreceptor (PR) cells, and an outer layer of RPE cells. Each of these layers can possess fully differentiated cells characteristic of the layer, and optionally can also contain progenitors of the differentiated cell characteristic of the layer. For example, the RPE cell layer (or core) can contain RPE cells and/or RPE progenitor cells; the PR cell layer can contain PR cells and/or PR progenitor cells; the inner nuclear layer can contain second-order retinal neurons and/or progenitors of second-order retinal neurons; and the RGC layer can contain RGCs and/or RGC progenitor cells. In some embodiments, the progenitor cells within the different layers described herein have the ability to complete differentiation following transplantation.

The terms “hPSC-derived 3D retinal tissue”, “hPSC-derived 3D retinal organoids”, “hPSC-3D retinal tissue,” “in vitro retinal tissue,” “hPSC-derived retinal tissue” “retinal organoids,” “retinal spheroids” and “hPSC-3D retinal organoids” are used interchangeably in the present disclosure and refer to pluripotent stem cell-derived three-dimensional aggregates comprising retinal tissue. The hPSC-derived 3D retinal organoids develop most or all retinal layers (RPE, PRs, inner retinal neurons (i.e., inner nuclear layer) and retinal ganglion cells) and display synaptogenesis and axonogenesis commencing as early as around 4-8 weeks in certain organoids and becoming more pronounced at around 3^{rd} or 4^{th} month of hESC-3D retinal development. The 3D retinal organoids disclosed herein may express the LGRS gene, which is an adult stem cell marker and an important member of the WNT pathway. In addition, the hPSC-derived 3D retinal organoids may be genetically engineered to transiently or stably express a transgene of interest to enhance differentiation and/or as a reporter and/or to enhance neuroprotective properties of hPSC-3D derived tissue constructs or cells derived from such tissue constructs.

Although the present disclosure refers to hESC-derived 3D retinal tissue, it will be appreciated by those skilled in the art that any pluripotent cell (ES cell, iPS cell, pPS cell, ES cell derived from parthenotes, and the like), as well as embryonic, fetal and/or adult retina, may be used as a source of 3D retinal tissue according to methods of the present disclosure.

As used herein, “embryonic stem cell” (ES) refers to a pluripotent stem cell (embryonic, induced or both) that is 1) derived from a blastocyst before substantial differentiation of the cells into the three germ layers (ES); or 2) alternatively obtained from an established cell line (iPS). Except when explicitly required otherwise, the term includes primary tissue and established cell lines that bear phenotypic characteristics of ES cells, and progeny of such lines that have the pluripotent phenotype. The ES cell may be human ES cells (hES). Prototype hES cells are described by Thomson et al. (Science 282:1145 (1998); and U.S. Pat. No. 6,200,806) and may be obtained from any one of number of established stem cell banks such as UK Stem Cell Bank (Hertfordshire, England) and the National Stem Cell Bank (Madison, Wisconsin, United States).

As used herein, “pluripotent stem cells” (pPS) refers to cells that may be derived from any source and that are capable, under appropriate conditions, of producing progeny of different cell types that are derivatives of all of the 3 germinal layers (endoderm, mesoderm, and ectoderm). pPS cells may have the ability to form a teratoma in 8-12 week old SCID mice and/or the ability to form identifiable cells of all three germ layers in tissue culture. Included in the definition of pluripotent stem cells are embryonic cells of various types including human embryonic stem (hES) cells, (see, e.g., Thomson et al. (1998) Science 282:1145) and human embryonic germ (hEG) cells (see, e.g., Shamblott et al.,(1998) Proc. Natl. Acad. Sci. USA 95:13726,); embryonic stem cells from other primates, such as Rhesus stem cells (see, e.g., Thomson et al., (1995) Proc. Natl. Acad. Sci. USA 92:7844), marmoset stem cells (see, e.g., (1996) Thomson et al., Biol. Reprod. 55:254,), stem cells created by nuclear transfer technology (U.S. Patent Application Publication No. 2002/0046410), as well as induced pluripotent stem cells (see, e.g., Yu et al., (2007) Science 318:5858); TakahasIn et al., (2007) Cell 131(5):861). The pPS cells may be established as cell lines, thus providing a continual source of pPS cells.

As used herein, “induced pluripotent stem cells” (iPS) refers to embryonic-like stem cells obtained by de-differentiation of adult somatic cells. iPS cells are pluripotent (i.e., capable of differentiating into at least one cell type found in each of the three embryonic germ layers). Such cells can be obtained from a differentiated tissue (e.g., a somatic tissue such as skin) and undergo de-differentiation by genetic manipulation which re-programs the cell to acquire embryonic stem cell characteristics. For example, induced pluripotent stem cells can be obtained by inducing the expression of Oct-4, Sox2, Kfl4 and c-Myc in a somatic stem cell. Thus, iPS cells can be generated by retroviral transduction of somatic cells such as fibroblasts, hepatocytes, gastric epithelial cells with transcription factors such as Oct-3/4, Sox2, c-Myc, and KLF4. Yamanaka S, Cell Stem Cell. 2007, 1(1):39-49; Aoi T, et al., Generation of Pluripotent Stem Cells from Adult Mouse Liver and Stomach Cells. Science. 2008 Feb. 14. (Epub ahead of print); 111 Park, Zhao R, West J A, et al. Reprogramming of human somatic cells to pluripotency with defined factors. Nature 2008; 451:141-146; K Takahashi, Tanabe K, Ohnuki M, et al. Induction of pluripotent stem cells from adult human fibroblasts by defined factors. Cell 2007; 131:861-872. Other embryonic-like stem cells can be generated by nuclear transfer to oocytes, fusion with embryonic stem cells or nuclear transfer into zygotes if the recipient cells are arrested in mitosis.

It will be appreciated that embryonic stem cells (such as hES cells), embryonic-like stem cells (such as iPS cells) and pPS cells as defined infra may all be used according to the methods of the present disclosure. Specifically, it will be appreciated that the hESC-derived 3D retinal organoids/retinal tissue may be derived from any type of pluripotent cells.

In an exemplary method for deriving 3-D retinal organoids, pluripotent cells (e.g., hESCs, iPS cells) are cultured in the presence of the noggin protein (e.g., at a final concentration of between 50 and 500 ng/ml final concentration) for between 3 and 30 days. Basic fibroblast growth factor (bFGF) is then added to the culture (e.g., at a final concentration of 5-50 ng/ml) along with noggin, and culture is continued for an additional 0.5-15 days. At that time, the morphogens Dickkopf-related protein 1 (Dikk-1) and insulin-like growth factor-1 (IGF-1) (each at e.g., 5-50 ng/ml) are added to the culture, along with the noggin and bFGF already present, and culture is continued for an additional time period of between 1 and 30 days. At this point, Dkk-1 and IGF-1 are removed from the culture and fibroblast growth factor-9 (FGF-9) is added to the culture (e.g., at 5-10 ng/ml) along with noggin and bFGF. Culture is continued in the presence of noggin, bFGF and FGF-9 until retinal tissue is formed; e.g., from 1-52 weeks. Additional examples of methods for deriving 3-D retinal organoids/tissues can be found in International Patent Application Publication No. WO 2017/176810, published on Oct. 12, 2017, which is incorporated by reference herein in its entirety.

In some embodiments, the organoids (hPSC-derived retinal tissue) may be disassociated prior to administration. The organoids may be disassociated at about 1 week, 2 weeks, 3 weeks, 4 weeks, 5 weeks, 6 weeks, 7 weeks, 8 weeks, 9 weeks, 10 weeks of development or culturing. In some embodiments, the organoids may be disassociated after 10 weeks of development or culturing. Organoids may be disassociated into their constituent cell types by suspension in solution or mechanically, with for example, a glass rod, a sieve, a blade, hydrophilic or hydrophobic surfaces, or any other appropriate means. According to certain embodiments, cell compositions are formulated from hESC-3D retinal tissue by dissociating the hESC-3D retinal tissue with papain.

The organoids or developing or differentiating organoids described herein may also be cultured and/or produced under non-adherent conditions or a combination of adherent and non-adherent conditions. In some embodiments, the organoids or developing organoids may be cultured on a substrate, manipulated, and subsequently cultured in non-adherent conditions. In some embodiments, the organoids may be cultured on a substrate, manipulated, and subsequently cultured in adherent conditions. In some embodiments, the organoids may be cultured in non-adherent conditions, manipulated, and subsequently culture in adherent conditions. In some embodiments the organoids, may be cultured in non-adherent conditions, manipulated, and subsequently cultured in non-adherent conditions.

In certain embodiments, the bioprosthetic retinal graft comprises hPSC derived organoids that have dimensions of between about 0.5 mm×0.5 mm to about 2 mm×2 mm. In other embodiments, the bioprosthetic retinal graft comprises hPSC derived organoids that have a diameter of between about 0.5 mm to about 2 mm.

In certain embodiments, proprietary lines of cGMP-grade hPSCs, which provide a replenishable source of stem cells tested in human ocular cell therapy trials, may be used.

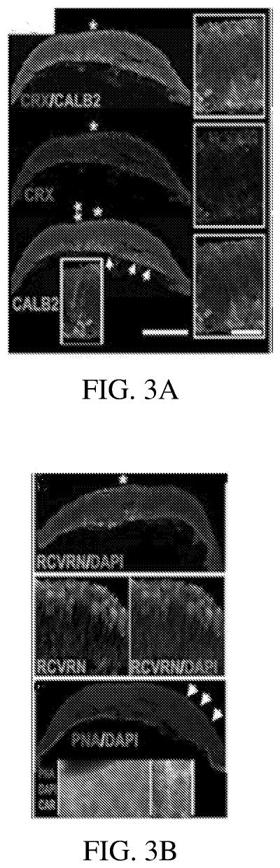

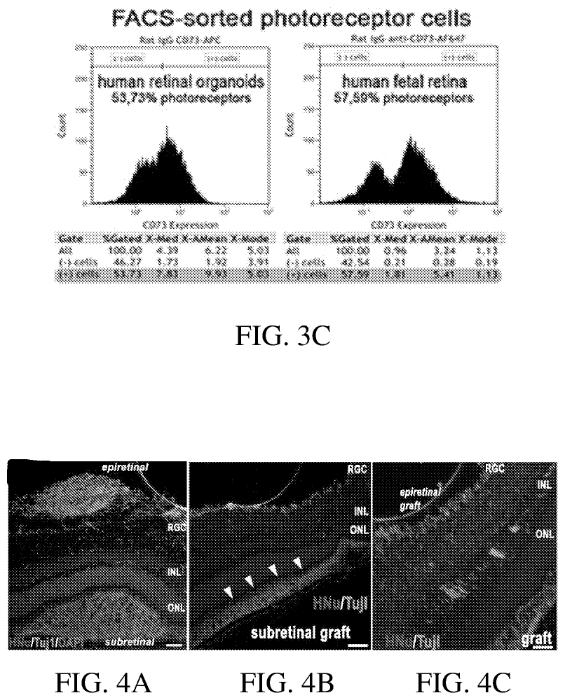

In some embodiments, the cell compositions which are suitable for therapeutic use may be formulated as cell therapy products comprising cryopreserved stocks of cGMP-grade human retinal progenitors, capable of delivering trophic support to degenerating retinal cells. Furthermore, retinal tissue from organoids derived in a dish is very similar to human fetal retina, as shown in FIG. 3A-FIG. 3C, with an almost identical percentage of photoreceptors (FIG. 3C) and is an excellent and replenishable source of primary human retinal progenitors. FIG. 3A shows images of hPSC derived retinal tissue stained with antibodies specific for the Calretinin marker, CALB2, which is expressed in neurons, including retina. FIG. 3B shows images of hPSC derived retinal tissue stained with antibodies specific for the retinal cytoplasmic marker, Recoverin (RCVRN).

In one aspect, the transplantable biological retinal prosthetic device comprises human pluripotent stem cell derived tissue (hPSC-3D retinal tissue or hPSC derived retinal tissue or organoids), human embryonic stem cells (hESC) and/or tissue, and/or human fetal retinal tissue or adult retinal tissue and a biocompatible carrier or scaffold to form a bioprosthetic retinal patch.

In some aspects, the biomaterial carrier or scaffold or matrix or delivery vehicle may be a structure such as, sheet, emulsion, network, slurry, or solution. In some aspects, the biomaterial carrier may be electrospun, printed, deposited, coated, lyophilized, or crosslinked. The biomaterial carrier or scaffold or matrix may contain multiple structures or traits, such as fibers, ridges, microneedles, and/or other architectural features. The biomaterial carrier may be comprised of biocompatible materials, such as polyphosphazenes, polyanhydrides, polyacetals, polyorthoesthers, polyphosphoesters, polycaprolactone, polyurethanes, polypeptides, polycarbonates, polyamides, polysaccharides, polyaminoacids, other polymers, proteins, metals, or ceramics. In some aspects the biomaterial carrier may be comprised in whole or in part of a derivation of a hyaluronan based hydrogel, such as HYSTEM® hydrogel (BioTime, Inc.). In some embodiments, a biomaterial carrier or scaffold may comprise combinations of the aforementioned traits and materials. In some embodiments, the carrier or scaffold may comprise thermo-reversible materials and/or shape memory metals. The scaffold (and bioprosthetic retinal patch) may be any shape suitable for delivery of hPSC tissue and/or cells and/or other components, such as exosomes or trophic factors.

The biological scaffold or support can comprise, for example, an electrospun polymer. In one embodiment, the electrospun polymer scaffold shares characteristics with Brunch's membrane. In some aspects, the thin electrospun nanofibers of biomaterial comprises a derivation of HYSTEM® hydrogel (BioTime, Inc.).

In some embodiments, biomaterial carriers or scaffolds may be used that have all of the characteristics required for successful delivery and/or securing in situ of complex, fragile cells and macromolecules.

Recently, a family of hyaluronan based hydrogels (trade named HYSTEM® and RENEVIA®) have been developed that mimic the natural extracellular matrix environment (ECM) for applications in 3-D cell culture, stem cell propagation and differentiation, tissue engineering, regenerative medicine, and cell based therapies. HYSTEM hydrogels were designed to recapitulate the minimal composition necessary to obtain a functional extracellular matrix. The individual components of the hydrogels are cross-linkable in situ, and may be seeded with cells prior to injection in vivo, without compromising either the cells or the recipient tissues.

The technology underlying HYSTEM® hydrogels is based on a unique thiol cross-linking strategy to prepare hyaluronan based hydrogels from thiol-modified hyaluronan and other ECM constituents. Building upon this platform, a family of unique, biocompatible resorbable hydrogels have been developed. The building blocks for HYSTEM® hydrogels are hyaluronan and gelatin, each of which has been thiol-modified by carbodiimide mediated hydrazide chemistry. Hydrogels are formed by cross-linking mixtures of these thiolated macromolecules with polyethylene glycol diacrylate (PEGDA) (see U.S. Pat. Nos. 7,928,069 and 7,981,871, incorporated herein by reference in their entirety). The rate of gelation and hydrogel stiffness can be controlled by varying the amount of cross-linker An attribute of these hydrogels is their large water content, >98%, resulting in high permeabilities for oxygen, nutrients, and other water-soluble metabolites.

Hydrogels, such as HYSTEM®, have been shown to support attachment and proliferation of a wide variety of cell types and tissues in both 2-D and 3-D cultures and exhibit a high degree of biocompatibility in animal studies when implanted in vivo. These hydrogels are readily degraded in vitro and resorbed in vivo through hydrolysis via collagenase and hyaluronidase enzymes. When implanted in these hydrogels, cells remain attached and localized within the hydrogel and slowly degrade the implanted matrix replacing it with their natural ECMs.

Crosslinkers may comprise, for example, a bi-, tri-, multi-functionalized molecule that is reactive to thiols (e.g. maleimido groups), oxidation agents that initiate crosslinking (e.g., GSSG), glutaraldehydes, and environment influences (e.g., heat, gamma/e-beam radiation). In some embodiments, there are no cross-linkers necessary.

Although specific examples of hydrogels that are suitable for providing resorbable matrices are described for use with embodiments of the present disclosure, it will be understood that any suitable biocompatible matrix may be used. For example, gels made using oxidized glutathione (GSSG) as a cross-linking agent may be used (see US Patent Application Publication No. US 20140341842, incorporated herein by reference in its entirety).

The carrier or scaffold may consist of decellularized tissue, such as retinal tissue. The decellularized tissue may be intact, disrupted, or manipulated, or may be mature tissue. The bioprosthetic retinal implant may consist, in whole or in part, of pieces of human embryoid retina, or fetal retinal tissue, or adult retinal tissue. May consist of organoid cells, or others, may consist of biomaterial. Or combo of these.

Because the compositions of cells, tissues and biocompatible carriers, matrices and scaffolds described herein elicit the proliferation of administered tissues, treatment results can be long lasting, such as, for example, greater than 18 months. In some embodiments, the carrier or scaffold is permeable to nutrients, trophic factors, and oxygen.

In some embodiments, the bioprosthetic carrier or scaffold can double as a cell culture and delivery substrate.

In some embodiments, the bioprosthetic retinal patch comprises the dimensions comprising a length×width×thickness of between about 0.5 mm×1 mm×1 μm and 8 mm×12 mm×100 μm. In some embodiments, the bioprosthetic retinal patch comprises a length×width×thickness of about 2 mm×4 mm×50 μm. In other embodiments, the bioprosthetic retinal patch comprises a length×width×thickness of about 4 mm×6 mm×10 μm. In some embodiments, the area of the bioprosthetic retinal patch comprises about 3 mm×6 mm, about 4 mm×6 mm, about 4 mm×5 mm.

In some embodiments, the bioprosthetic retinal graft or patch may be anchored after implantation using any material suitable.

In one aspect, the retinal tissue and biocompatible scaffold are joined together by a biocompatible adhesive.

In another aspect, the cell therapy is formulated according to a method comprising imbedding organoid pieces into a biocompatible scaffold, wherein the biocompatible scaffold is initially formulated in a liquid form and then forms a gel, and wherein prior to complete solidification, the pieces are placed in the liquid scaffold such that when the scaffold gels, the organoid pieces become imbedded in the gel. In one embodiment, the graft can be administered prior to complete gelation of the scaffold. In another embodiment, the graft can be administered in a suspension of biomaterial or in conjunction with a biomaterial or biocompatible adhesive or a combination thereof.

In some embodiments, organoids may be crosslinked to a biocompatible scaffold using natural proteins or small molecule crosslinkers, such integrins or fibronectins. In some aspects, several pieces of retinal tissue are fastened or adhered to a large biomaterial scaffold to create a large retinal implant or biological retinal prosthetic device.

In some embodiments, organoids may be modified to increase their adhesion to the carrier, substrate, or recipient tissue.

In some aspects, several pieces of retinal tissue are fastened or adhered to a thin film of biomaterial to create an implant or biological retinal prosthetic device, as shown in FIG. 1C. In some aspects, the thin film of biomaterial may comprise biological components, such as a layer of RPE, an RPE sheet, RPE cells, progenitor cells or cell types other than those that comprise the organoids, as shown in FIG. 1D.

In some aspects, the organoids or biological components may be cultured or adhered to a non-biodegradable carrier or scaffold which is enzymatically dissolved, and the retinal tissue and/or other biological components attached to biodegradable carrier or scaffold and implanted.

In certain embodiments, the retinal tissue and biological scaffold may be described as an implant. In certain embodiments, the retinal tissue and biocompatible carrier or scaffold may be described as a medical device or biological retinal prosthetic device.

In some aspects, multiple three-dimensional (3D) retinal tissue pieces each carrying between about 1,000 to 2,000 or 2,000 to 3,000, or 1,000 to 5,000, 3,000 to 10,000, or 5,000 to 100,000, or 50,000 to 500,000 or 100,000 to 1,000,000 photoreceptors can be mounted on a thin or ultrathin flexible biomaterial to capture and synaptically (or by other means) transmit visual information to a subject's RGCs, which will then be conducted to the subject's visual cortex. The total implanted tissue pieces can produce a patch or biological retinal prosthetic device with between approximately 1,000 to 2,000 or 2,000 to 3,000, or 1,000 to 5,000, 3,000 to 10,000, or 5,000 to 100,000, or 50,000 to 500,000 or 100,000 to 1,000,000 or more individual light sensors, i.e. photoreceptors, capable of creating a wide visual angle (up to 30° depending on the dimensions of the biological retinal patch) to support useful, functional vision. By comparison, the Argus II neuroprosthetic device has only 60 sensors, which only allows a recipient to discern the shapes of objects, when positioned accurately into subretinal space.

In some embodiments, organoids may be combined with synthetic materials, sensors, chips, or electronic devices. In one embodiment, a bioprosthetic retinal patch is described comprising, hPSC derived retinal tissue and a film or biological scaffold or matrix comprising a biocompatible material with photosensitive diodes (photodiodes) to form a photosensitive component or layer. The hPSC derived retinal tissue or organoids are combined with or adhered to the photosensitive layer using any of the materials and methods described herein. FIG. lE shows an illustration of a bioprosthetic scaffold with photodiodes. The photodiode layer can enhance the response to light (capturing light, converting light into electric signals and transmitting the signals) by the host's remaining functional photoreceptors and retinal tissue component of the patch, especially in the areas of the retinal graft tissue is still developing or differentiating.

In other embodiments, a large graft comprising many pieces of hESC-3D retinal tissue and a biocompatible scaffold is engrafted into the subretinal space of a subject resulting in tumor free synaptic integration. In some embodiments, the biocompatible scaffold is porous to allow for easier synaptic connections and transfer of molecules between cells and cell layers.

Therapeutic targets of such technology are human RD conditions, associated with PR death and blindness, such as but not limited to, Retinitis Pigmentosa (RP), and Age Related Macular Degeneration (AMD). Cone-only hPSC-3D retinal tissue from retinal organoids may also be derived to treat disorders and diseases, such as AMD. Bionic chips (e.g., SecondSight, ARGUS® II, 60 pixels) work in a similar way, though biological design can outperform electronic design due to limitations of electronics and the transient life span of grafted electronic chips. A biological retinal patch is integrated with the host's tissues, brings thousands of PRs (i.e., pixels) per single slice of retinal organoid and can be tailored (constructed) to treat individual diseases.

In certain embodiments, ocular grafting may be carried out by any acceptable methods, including for example, the methods described in International Patent Publication No. WO2016/108219, incorporated herein by reference in its entirety.

In other embodiments, ocular grafting can be carried out by a mechanical motorized delivery device, such as the UMP3 UltraMicroPump III with Micro4 Controller (World Precision Instruments), or a variation thereof, according to manufacturer's instructions.

In certain embodiments, the delivery device may comprise a canula. The canula can comprise an inner diameter of between about 0.5 mm to about 2.5 mm or about 1 mm to about 2 mm or about 1.12 mm. The canula may also comprise an outer diameter of between about 0.5 mm to about 3 mm, or about 1 mm to about 2.5 mm or about 1.25 mm to about 1.5 mm or about 1.52 mm.

In certain embodiments, the bioprosthetic retinal graft or patch may be delivered to a subject's ocular space using a cannula, whereby air bubbles are introduced into the cannula before and/or after the bioprosthetic retinal graft or patch, as shown in FIG. 1G, in order to prevent the bioprosthetic retinal graft or patch from exiting the cannula before it is in position. In certain embodiments, intraocular pressure may be applied to the subject's eye at the same time the bioprosthetic retinal graft or patch is implanted in order to assist in keeping the bioprosthetic retinal graft or patch in place after implantation. In another embodiment, epinephrine may be injected into the vitreous space to suppress bleeding that may occur as a result of administering the bioprosthetic retinal graft or patch using a procedure that requires an incision, such as retinotomy.

In certain embodiments, surgical procedures may comprise but are not limited to, vitrectomy, relaxed vitrectomy, relaxed retinotomy, the use of retinal tacks, retinal detachment and macular translocation. Relaxing retinotomy, which allows a large piece of patient's retina to be peeled off and then reattached, has been used in clinic. These surgical techniques can be repurposed for placing a large bioprosthetic retina into the subretinal space of a subject, enabling a large area of a subject's eye to regain visual perception. In certain embodiments, adhesives, staples or any other material suitable for aiding in the administration or fixation of the bioprosthetic retinal grafts and patches described herein and/or the healing of surgical wounds may be used.

In certain aspects, the bioprosthetic graft or patch can be rolled or otherwise compressed in order to fit into a smaller incision (about 3 mm or less). The graft or patch may then unroll or expand back to its original shape in situ, as shown in FIG. 1F. In some embodiments, the graft or patch can return to its original shape without further surgical intervention or manipulation, once implanted within the subject's eye. In some embodiments, the graft or patch can return to its original shape on its own without further manipulation within between about 2 to 15 seconds after implantation. In certain embodiments, the graft or patch may be pre-loaded and/or stored in the delivery device for a period of time before delivery into the subject's eye.

In certain embodiments, several bioprosthetic retinal grafts or patches may be loaded into a delivery device comprising a delivery component such as a cannula, for example, and administered into the ocular space one after another, to cover a large area.

Aspects of the present disclosure provide a robust vision restoration therapy for patients, especially those patients whose retina is too damaged to be preserved by neuroprotection alone, wherein individual photoreceptors can permanently wire synaptically onto a recipient's ganglion cells and/or other retinal or support cells and create a large visual angle restoration or amelioration of vision within 12 months after grafting. This vision restoration method is efficient and permanent due to synaptic wiring of individual sensors (photoreceptors) onto a subject's RGCS. By contrast, subretinally implanted synthetic neuroprosthetic devices gradually lose contact with the RGCS in retinal injuries where the retina remains by and large intact, but susceptible to gradual irreversible degeneration following, for example, a blast injury or degenerative disease.

As used herein, the term “synaptic activity” or “synaptically” refers to any activity or phenomenon that is characteristic of the formation of a synapse between two neurons.

Evaluation of the therapeutic effects of the bioprosthetic graft and methods for making bioprosthetic grafts described herein can be measured, for example, by (at selected time points after a blast injury, for example) an increase in the Visually Evoked Potential (VPE), a reliable method to evaluate the intensity of a visual signal reaching the brain. Electroretinography, multifocal ERG, multielectrode array (MEA) and/or RetiMap method may also be used.

In some embodiments, use of advanced methods of evaluating synaptic connectivity between the graft (hPSC-3D retinal tissue and/or cell, etc.) and/or bioprosthetic retinal patch (hPSC-3D retinal tissue and/or cells, etc. and a biocompatible carrier or scaffold) and the recipient retina, such as the genetic transsynaptic tracer, WGA-HRP (expressed by the transplant but not the recipient retina), WGA-Cre, human SYP, SC121 antibodies or immuno-electron microscopy are provided to demonstrate the chimeric (graft:recipient) synaptic connectivity. This tracing may not only improve mapping of graft/host connections but can also distinguish cell fusion and neuroprotection from specific synaptic integration.

In some embodiments, large eyed animal models, such as the Pde6a−/−dog, Aipl—/—cat, Cngb3-mutant dog and Crx-mutant [+/−] cat, an Aipl-1 mutant cat, or rabbits with ocular blast injury may be used to demonstrate efficacy of the hPSC-3D retinal tissue or hPSC-3D bioprosthetic retinal implant/grafts, each of which have PR degeneration, retinal degeneration and/or optic nerve degeneration similar to that of human subjects with genetic retinal degeneration conditions, retinal diseases or injury.

In some embodiments, in vivo readout approaches may be used to evaluate the extent of vision restoration after transplantation of hPSC-3D retinal tissue into the subretinal space of a subject, including but not limited to, full-field ERG, multifocal ERG microelectrode array (MEA), pupil imaging and visual evoked potential (VEP), in addition to behavioral tests.

In some embodiments, a subretinal graft of hPSC-3D retinal tissue (retinal organoid; bioprosthetic retinal implant/patch) may act as a biological analog of a neuroprosthetic device, which can capture visual information and synaptically transmit it to retinal ganglion cells and then to the visual cortex. In another embodiment, the implant supports restoration of visual perception (light detection) in a subject.

In yet other embodiments, hPSC-derived retinal organoid bioprothetic implants/patches or biological retinal prosthetic devices carrying a layer of PRs and second order neurons provide the light sensors that can synaptically transmit visual information to a subject's RGCs, which persist even after all PRs are degenerated. Unlike electroprosthetic chips, a “bioprosthetic” implant based on hPSC-derived retinal organoids can enable long-lasting synaptic integration and can be adjusted to carry more cones than rods to repair and rebuild the macula. In some embodiments, long-term restoration of light sensitivity can be seen in a majority of the subjects using subretinally grafted hPSC-3D retinal tissue.

In some embodiments, synaptic connectivity and functional integration of hPSC-3D retinal tissue grafts into the retinal circuitry of a subject and can be demonstrated using preembedding immunoEM, electroretinogram recording and multielectrode-array recording.

In some embodiments, tumor-free survival of grafted hESC-3D retinal tissue in the subretinal space occurs for at least about 6 to 24 months, with lamination and development of PR and RPE layers, including elongating PR outer segments, synaptogenesis, electrophysiological activity and connectivity with the recipient retinal cells, and development into more mature retinal immunophenotypes. In some embodiments, hESC-3D retinal tissue grafts improve visual perception in subjects within about 5 to 10 months after grafting due in part to gradual maturation and synaptic integration. In some embodiments, cytoplasmic fusion between the graft and the host in addition to specific synaptic connectivity between the graft and the host, is demonstrated.

Fetal retina grafting into the subretinal space of visually impaired patients has been shown to improve vision in up to 7 out of 10 cases. Though it may be reasonably argued that the fetal retina grafts positively impacted the patient's degenerating retina via neuroprotection mechanisms, there is also evidence for specific synaptic connectivity established between the graft and the recipient retina. In both RD rats and RD patients, human fetal retinal grafts were found to improve visual responses (superior colliculus activation in rats, visual acuity improvements in patients [ClinicalTrials.gov ##NCT00345917, NCT00346060]).

Similarly, hPSC-3D retinal tissue of the present disclosure has been shown to enable light-evoked superior colliculus responses in blind RD rats with no functional PRs, indicating that PRs in the graft transmitted visual information to the brain. In addition, there is evidence that hPSC-3D retinal organoids develop the inner/outer segments and cilia of PRs in subretinal grafts, even though such grafts did not maintain continuous laminated structure. The hPSC-3D retinal tissue is very similar to human fetal retina, displays robust synaptogenesis and electrical activity after about 6 to 8 weeks of development, and contains rudimentary inner segment-like protrusions immunopositive for peanut agglutinin (PNA), which collectively indicate that once the tissue is subretinally transplanted it will be ready for further development, maturation and synaptic integration. Consequently, there is evidence provided herein of graft/host connectivity in hPSC-3D retinal tissue grafted in the subretinal space of immunosuppressed wild-type cats. Taken together, these data indicate that hPSC-derived 3-D tissue and bioprothetic grafts can restore retinal photosensitivity in at least the area receiving the graft.

An advantage of this approach is the ability to derive human fetal-like retinal tissue carrying its own layer of RPE. This RPE layer can assist in the survival of hPSC-3D retinal tissue after grafting. The competing technologies can generate a neural retinal layer but not RPE from hPSC cultures. Neural retina and RPE develop together, induce each other to promote structural and functional maturation in development and depend upon each other to carry out visual function. Grafting hPSC-derived neural retina without a RPE layer can deprive developing PRs of paracrine and structural support from the RPE. There may be a gap in the subretinal space between the RPE layer of the recipient retina and PRs of the graft. Lack of physical interaction between the microvilli of RPE and developing PRs can interfere with the apical RPE's ability to induce PR outer segment elongation. Alternatively, hPSC-3D retinal tissue derived by the methods described herein does not depend on the close proximity to the recipient's RPE and will have advanced survival and differentiation (as an independent patch) in subretinal grafts. This, in turn, increases the ability of hESC-3D retinal tissue patches to restore visual function. There is evidence that retina+RPE grafted together leads to better vision improvement in RD patients. However, these pilot trials used human fetal retinal tissue, which cannot be used for routine treatment due to ethical restrictions and tissue availability. Human ES cells provide a limitless source of cells for derivation of retinal tissue. Accordingly, the hPSC-3D retinal tissue grafts of the present disclosure overcome two major obstacles to treatment of retinal degenerative diseases and injuries: availability of human fetal retina, and ethical restrictions.

To enable a retina with degenerated PRs to regain light perception, a new set of “sensors” is needed, which are able to be electrically connected to the remaining retina of a subject to enable the transmission of the electric signals. Human ESC-derived retinal tissue (retinal organoids, size 0.3-0.5 mm length) is similar (histologically, and based on marker expression) to human fetal retina, and develops layers of RPE, PRs, second order retinal neurons and RGCs between week 6-8 of development in vitro, when growing as substrate-attached aggregates. The hPSC-3D retinal tissue develops axons (especially RGC-specific long axons) and multiple synaptic boutons by 6-8 weeks of development, when growing as substrate-attached aggregates. Also, this hPSC-3D retinal tissue can become progressively electrically active between week 8 and week 12 of in vitro development. A piece of retinal organoid grafted into the subretinal space can bring a sufficient number of PRs to enable a blind animal to regain light perception.

Neurotrophic factors are a diverse group of soluble proteins (neurotrophins), and neuropoietic cytokines, which support the growth, survival and function of neurons. They can activate multiple pathways in neurons, ameliorate neural degeneration, preserve synaptic connectivity and suppress cell death in retinal tissues. Acutely injured retina will survive if neuroprotection is provided in the form of small molecules, neuroprotective proteins such as Brain-Derived Neurotrophic Factor (BDNF) or cells and delivered efficiently and early enough to suppress cell death and/or initiation of retinal remodeling and scarring. However, if degeneration proceeds unabated without treatment, progressive vision loss can be expected due to the loss of photoreceptors, RGCs and other retinal neurons as well as retinal remodeling and scarring.

The Retina is a very delicate thin layer of neural tissue, which receives light stimulation and converts it to electrical impulses, transmitted via the optic nerve to the brain (lateral geniculate nucleus) and eventually to the visual cortex. The optic nerve originates in the retina and is formed by the axons of retinal ganglion cells (RGCs), one of the seven cell types found in retinal tissues. Contusion injury is caused when the globe is initially compressed by the blast force and then rebounds to normal shape but overshoots and stretches beyond its normal shape. Nonpenetrating globe injuries are, therefore frequent on the battlefield and may result in retinal trauma such as, for example, retinal detachment, optic nerve damage, retinal remodeling, axonal deafferentation (the disruption of the afferent connections of nerve cells), which often leads to slow (up to several months) cell death and progressive vision loss, even though retinal structure may be initially preserved.

In some embodiments, hESC derived retinal tissue grafts are capable of delivering neurotrophic factors and/or mitogens after implantation. In some embodiments, the hESC derived retinal grafts or patches comprising dissociated cells of the hESC derived retinal tissue are also capable of delivering neurotrophic factors and/or mitogens after implantation. In some embodiments, the hESC derived retinal tissue and/or cells are capable of delivering neurotrophic exosomes to a subject after implantation. The neurotrophic factors and mitogens in which the grafts described herein are capable of delivering to a subject include but are not limited to, brain-derived neurotrophic factor (BDNF), glial-derived neurotrophic factor (GDNF), neurotrophin-34 (NT34), neurotrophin 4/5, Nerve Growth Factor-beta (βNGF), proNGF, PEDF, CNTF, pro-survival mitogen basic fibroblast growth factor (bFGF=FGF-2) and pro-survival members of the WNT family.

Current military standard of care for eye injury caused by traumatic or blast overpressure injury is to employ the Birmingham Eye Trauma Terminology System (BETTS) and Ocular Trauma Classification Group to determine appropriate treatment (see FIG. 2). Blast injuries are generally attributed to four mechanisms: the primary blast (overpressure impulse); secondary effects such as penetrating wounds caused by shrapnel blown about by the blast forces; tertiary injuries caused by, for example, the individual being thrown forcefully against a rigid structure; and quaternary injuries caused by ancillary processes such as toxic fumes, chemical burns, or even long-term psychological effects (Morley et al. 2010). Closed globe trauma is subdivided into zones, each with unique injury patterns: Zone I includes the conjunctiva and corneal surface; Zone II includes the anterior chamber, lens, and pars plicata. Zone III includes the retina and optic nerve. Each of the Zones is illustrated in FIG. 3.

There are some tested guiding principles which govern the responses of retina/optic nerve to high-pressure blast injury. If the primary damage to Zone III is retinal detachment, this will initiate rapid apoptosis of the photoreceptor layer in the days to weeks post injury, followed by degeneration of the inner nuclear layer (INL), retinal remodeling, vision distortion and loss of vision. However, the retinal ganglion cell (RGC) layer will survive for months to years post injury as long as there is preservation of axonal connectivity between the RGC nerve fibers (forming the optic nerve) and the neurons of the visual cortex.

RGC viability depends on their connectivity to visual cortex neurons, and such afferents carry supportive (trophic) factors between RGCs and visual cortex neurons. Blast exposure can cause deafferentation and therefore disrupt the flow of trophic factors leading to the gradual but steady loss of vision. Restoration of trophic support (even partial) leads to preservation of RGCs. Several trophic factors administered together can produce a potent neuroprotective defense against RGC apoptosis after axotomy. Therefore, it is helpful in the days to weeks following injury to administer treatment to preserve RGCs after loss of connectivity.

Photoreceptor viability may be partially dependent upon trophic support, for example, from the retinal pigment epithelium (RPE) and synaptic contacts with inner nuclear layer (INL) neurons. Photoreceptor viability and function depend on RPE-photoreceptor connectivity. Retinal detachment after blast injury results in degeneration of photoreceptor outer segments. The time frame in which photoreceptor function can be restored after reattachment is usually in the days to weeks post injury. As shown herein, restoration of trophic support to photoreceptor cells (even partial) leads to long-term preservation of photoreceptors.

Efficient treatment of vision problems associated with ocular blast injury requires an understanding of the neuropathology of damage caused by blast injury to the visual system. Though the initial damage may not be immediately apparent, the blast pressure wave causes elongation and/or splitting of cells and axonal shearing in the direction of wave propagation, leading to the slow degeneration of the retina and the optic nerve. The polytrauma nature of combat injuries often leads to competing priorities of care. While top concerns on the battlefield are blood loss and resuscitation, after stabilization, attention can turn to ensuring the best possible outcomes for all injuries. Initiation of ophthalmic care often occurs in the hours to days after injury. This treatment window falls well within the timeline thought to enable an effective treatment option for closed globe ocular injury. Preserving the original neural architecture of retina, required for visual function, and preventing retinal degeneration after blast injury (by neuroprotection) is a feasible therapeutic mechanism in which to ameliorate blindness.

Accordingly, in one embodiment, cell compositions formulated from hPSC-3D retinal tissue (hESC-3D retinal organoids) which are suitable for therapeutic use are obtained and transplanted into a subject's ocular space, wherein the cells are capable of secreting neurotrophic factors, mitogens and/or extracellular components, such as exosomes. In some embodiments, the cell compositions continuously deliver (by secreting or other mechanism) trophic factors during the appropriate treatment window. According to some embodiments, the cell compositions deliver (by secreting or other mechanism) a combination of several trophic factors mitogens and/or extracellular components, such as exosomes simultaneously. In another embodiment, the trophic factors mitogens and/or extracellular components, such as exosomes produced by the bioprosthetic retinal grafts or patches grafted into the ocular space (e.g., subretinal or epiretinal) can provide a potent neuroprotective defense against retinal cell death. The therapeutic targets may include some or all cell types of the subject's retina (e.g., photoreceptors, RPE, second order neurons, RGCs/optic nerve).

In some embodiments, the therapeutic impact is enhanced by transplanting cell compositions comprising RPE cells, retinal ganglion cells (RGCs), second-order retinal neurons (corresponding to the inner nuclear layer of the mature retina), and photoreceptor (PR) cells. The therapeutic effect may be enhanced by the combination of neuroprotection from the transplanted cells. In other embodiments, different cell types may be sorted and isolated in order to create a higher concentration of a particular cell type and consequently higher concentrations of specific tropic factors in order to treat a specific disease, injury or condition.

Stem cell-derived grafts described herein can provide long-lasting trophic support to degenerating retinal neurons and are thus a broadly applicable treatment modality for ocular blast injury. Retinal cell grafts may alleviate vision loss after sustained blast injury to Zone III (retina-optic nerve-visual cortex).

In one embodiment, grafts of stem cell-derived human retinal progenitor cell compositions are formulated to exert strong neuroprotective support on rabbit neural retina and the optic nerve, damaged by CIS 2-3 blast injury, which can ameliorate vision loss. Functional integration of some grafted neurons may further protect the retina from degeneration and positively contribute to vision preservation.

In other embodiments, the cell compositions or stem cell-derived grafts can provide long-lasting trophic support to degenerating retinal neurons and thus provide a feasible and broadly applicable therapeutic intervention to attenuate vision loss caused by ocular blast injury. The cell therapy compositions described herein are capable of positively affecting the preservation of photoreceptors and retinal ganglion cells (RGCs).

According to certain embodiments, therapeutic cell compositions described herein provide efficient, controlled and continuous paracrine delivery of a cocktail of neurotrophic factors into the damaged retinal tissue. The therapeutic cell compositions described herein can be particularly effective in retinal injuries where the retina remains by and large intact, but susceptible to gradual irreversible degeneration following blast injury due to a disruption of the of the highly ordered tissue architecture.

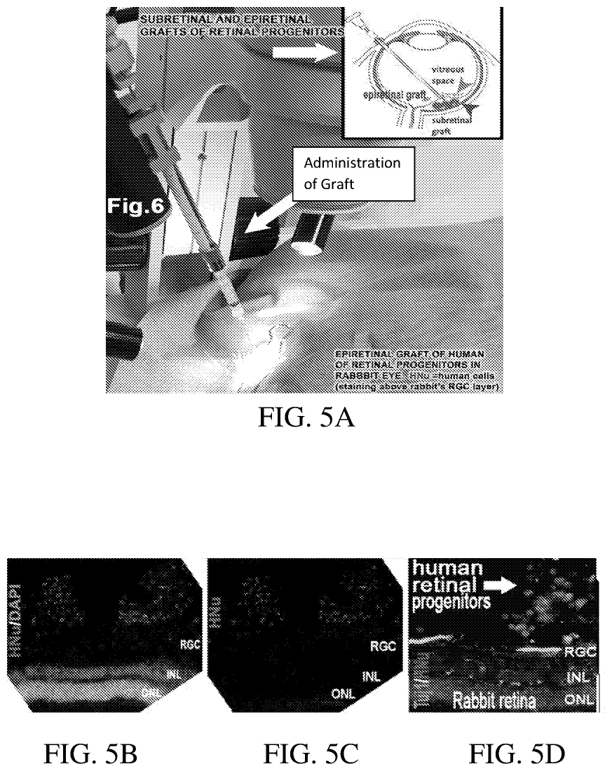

FIG. 5B through FIG. 5D demonstrates that that subretinal grafts of human retinal progenitors differentiated from human embryonic stem cells (hESCs) can be successfully transplanted into the ocular space of a large eyed animal model (rabbit), can preserve the thickness of retinal layers in adult mammalian retina for up to 3 months, have no deleterious impact on recipient retina, and do not cause tumorigenesis. Cells from these grafts migrate and integrate into recipient retinal layers, thus strengthening the recipient retina. Such cells intermingle with recipient retinal cells in RGC and INL and can exert paracrine support to the host cells around them. FIG. 4A shows an ICH image of retinal integration and maturation of hESC derived retinal progenitor cells (hESC-RPCs) transplanted into the epiretinal space of a mouse model. As shown, most of the human progenitor cells are negative for the early neuronal marker, Tuj1, and can be seen migrating and integrating into the host's retinal ganglion cell (RGC) layer or inner nuclear layer (INL). FIG. 4B shows an ICH image of implanted hESC derived retinal progenitor cells migrating over a large area of the host's subretinal area. FIG. 4C shows an ICH image of cells from implanted epiretinal hESC-RPCs integrating into the host's retinal ganglion cell (RGC) layer, inner plexiform layer, and inner nuclear layer (INL). Cells deposited into subretinal and epiretinal space can migrate out into the host retina, without leaving any bulging in the subretinal space or epiretinal membrane on top of the RGC layer.

In one embodiment of the present disclosure, the neuroprotection from transplanted cells on retina impacted by blast injury increases cell viability and/or cell survivability by between about 10% and about 250% compared to cell viability of control retina.

The cell compositions described herein are suitable for therapeutic use in sustaining the viability and visual function of the retina, optic nerve and visual cortex following retinal detachment and optic nerve damage from closed globe wounds or disease. As the technology does not require an autologous donor cell source, therapeutic cells can be made available on demand for the treatment of ocular trauma, disease and vision loss.

In some embodiments, 80 percent of subjects have retinal cells surviving in sub/epiretinal space after grafting by 3-6 months. In another embodiment, 80 percent of subjects with retinal grafts found by OCT (total of ˜64% of total subjects) will have improved VEP and ERG results by 1 month, 2 months, 3 months, 4 months, 5 months, or 6 months after ocular grafting of a bioprosthetic retinal graft or patch due at least in part to neuroprotection from retinal progenitors.

In one embodiment, preservation of retinal thickness in subjects will occur by between about 1 to about 6 months after grafting. In another embodiment, subjects will have reduced cell death at or near the graft, as assessed by for example, Cleaved Caspase-3, γH2AX (early apoptosis markers) and Tunnel staining (late marker)).

In yet another embodiment, preservation of retinal thickness (as a key readout for retinal degeneration) in at least about 64% of subjects will occur between about 1 to about 6 months after grafting, and reduced cell death as assessed by for example, (Cleaved Caspase-3, γH2AX (early apoptosis markers) and Tunnel staining (late marker).

Subretinal grafts can provide neuroprotection on photoreceptors and outer plexiform (synaptic) layer, while epiretinal grafts can neuroprotect RGCs/optic nerve, second order retinal neurons and inner plexiform (synaptic) layer.

In one embodiment, subjects presented retinal thickness preservation of about 1% to about 15% at about 6 months after grafting of the bioprosthetic graft.

In certain embodiments, therapeutic cell compositions are administered with or without immunosuppression.

The retina is an intricate structure and preservation of cells and synaptic networks helps to maintain vision. Restoring the original neural architecture of the retina helps to alleviate diseases such as retinitis pigmentosa and AMD.

### EXAMPLES

The following examples are not intended to limit the scope of what the inventors regard as their invention nor are they intended to represent that the experiments below are all or the only experiments performed.

### Example 1

Restoration and improvement of visual perception will be demonstrated in rabbits with ocular blast exposure and retinal damage. Subretinal grafts comprising hESC-3D retinal tissue alone (without biomaterial/scaffold) will be used to treat damaged retinal tissue in rabbits. Structural restoration of tissue and vision will be demonstrated using optical coherence tomography (OCT) in live animals and histology and immunohistochemistry after sacrificing. Functional restoration will be demonstrated using visual evoked potential (VEP) in live animals.

Human retinal tissue is generated using clinical-grade hPSCs (BIOTIME, INC.). A pilot grafting experiment in rabbits will be performed to determine the subretinal grafting procedure in a large eye animal model. Ocular blast injury models are generated in rabbits using a shock tube. Multiple pieces of hESC-3D retinal tissue (between about 0.1 and about 1 mm length) are then transplanted into the subretinal space of each animal.

Ocular blast injury models may include those described in Gray, W., Sub-lethal Ocular Trauma (SLOT): Establishing a standardized blast threshold to facilitate diadnostic, early treatment, and recovery studies for blast injuries to the eye and optic nerve. Final report, prepared for: U.S. Army Medical Research and Material Command. Award Number: W81XWH-12-2-0055, 2015, for example.

Structural integration of retinal tissue is evaluated by OCT, and functional integration/improvement of visual perception is evaluated by measuring VEP at 1, 2, 3, 4, 5 and 6 months after surgery. Both eyes of each animal are used for grafting of retinal tissue, and VEP is evaluated independently for each eye by covering the counterpart eye.

The following controls may be used: control, 1 eye (no treatment), control 2, counterpart eye (sham-treatment, i.e., grafted with biomaterial only, no organoids).

Implanted hESC-3D retinal tissue grafts can synapse on a rabbit's RGCs and/or second order retinal neurons, which can enable the animal to regain visual perception by between about 4 to 6 months after surgery (as measured by a VEP signal) Similar dynamics were observed in a blind rat animal model, which received hESC-3D retinal tissue grafted in subretinal space.

Cohorts can comprise between 8 and 15 rabbits. Accordingly, statistical analysis can be performed (1-way ANOVA).

### Example 2

Restoration and improvement of vision will be demonstrated in rabbits with ocular blast exposure and retinal damage. Subretinal grafts comprising hESC-3D retinal tissue and a biodegradable and/or non-biodegradable carrier or scaffold will be used to treat damaged retinal tissue in rabbits. The subretinal grafts may comprise hESC-3D retinal tissue pieces mounted on a thin layer of electrospun nanofibers of biomaterial scaffold to form a biological retinal patch, as described herein. Structural restoration of tissue and vision will be demonstrated using optical coherence tomography (OCT) in live animals and histology and immunohistochemistry after sacrificing. Functional restoration will be demonstrated using visual evoked potential (VEP) in live animals.

Human retinal tissue is generated using clinical-grade hPSCs (BIOTIME, INC.). A pilot grafting experiment in rabbits will be performed to determine the subretinal grafting procedure in a large eye animal model. Ocular blast injury models are generated in rabbits using a shock tube. Multiple pieces of hESC-3D retinal tissue (between about 0.1 and about 1 mm length) with a biodegradable carrier or scaffold are then transplanted into the subretinal space of each animal.

Hydrogels (such as those derived from hyaluronic acid, alginate, etc.) may be used as the biodegradable carrier or scaffold, for example. Hydrogels can be formulated to gel in situ in the subretinal space in between about 1 minute to about 60 minutes after grafting and can secure the grafted pieces of retina in the subretinal space, thereby improving surgical and functional outcomes. This study will demonstrate that transplanting hPSC-3D retinal tissue pieces together with biodegradable biomaterial can improve the surgical and functional outcome of the procedure, leading to more animals with an increase in VEP signal between 4-6 months post-surgery.

A biological retinal patch or biological retinal prosthetic device is constructed with several pieces of hPSC-3D retinal tissue mounted on a patch of very thin biomaterial (approximately between 3-5 mm wide and 5-8 mm long) to support transplantation into subretinal space of rabbits with ocular blast injury.

During administration, the biological retinal patch may be placed in the retinal space with the retinal tissue positioned for maximum vision restoration. The retinal patch can be administered so that the patch is stabilized within a retinal bleb created prior to administration of the retinal graft or patch. The implant may be affixed with a complementary material or procedure.

### Example 3

hPSC-retinal progenitors were delivered into the ocular space of rabbits (ex vivo experiments), using an ocular injector. The frozen sections of rabbit eyes grafted with human retinal progenitors were stained with anti-human nuclei antibody HNu (red) and pan-nuclei DAPI stain (blue). The presence of human retinal cells (red+blue stain) in the rabbit's ocular space (blue stain only), delivered with the help of the ocular injector, was demonstrated. FIG. 5B through FIG. 5D demonstrate that that subretinal grafts of human retinal progenitors differentiated from human embryonic stem cells (hESCs) can be successfully transplanted into the ocular space of a large eyed animal model (rabbit), can preserve the thickness of retinal layers in adult mammalian retina for up to 3 months, have no deleterious impact on recipient retina, and do not cause tumorigenesis. Cells from these grafts migrate and integrate into recipient retinal layers, thus strengthening the recipient retina. Such cells intermingle with recipient retinal cells in RGC and INL and can exert paracrine support to the host cells around them.

### Example 4

### Cells of hPSC-3D Retinal Tissue Secrete Neurotrophic Factors

The conditioned medium from hPSC-3D retinal tissue cultures (and conditioned medium from undifferentiated hESCs as a control) were assayed for the presence of several key trophic factors such as brain-derived neurotrophic factor (BDNF), glial-derived neurotrophic factor (GDNF), neurotrophin-4 (NT4), Nerve Growth Factor-beta (βNGF) and pro-survival mitogen basic fibroblast growth factor (bFGF=FGF-2). The Luminex technology (RnD Systems) was used to read the concentration of these neurotrophic factors and high levels of BDNF and GDNF were found, in addition to bFGF in conditioned medium, exceeding the control level of undifferentiated hESCs by at least between about 100 fold 1,000 fold, resulting in picoograms to nanograms/ml concentration of neurotrophins.

### Example 5

### Rabbit Blast Ocular Injury Model

A rabbit blast ocular injury model based on Jones, K., et al., Low-Level Primary Blast Causes Acute Ocular Trauma in Rabbits. J Neurotrauma, 2016. 33(13): p. 1194-201 was designed to evaluate the potential of cell preparations described herein to ameliorate retinal degeneration and optic nerve damage caused by blast injury to alleviate or halt vision loss. The two routes of cell delivery are (i) epiretinal, and (ii) subretinal to find the route leading to the greatest survival, and the most efficient retinal integration of grafted cells, that collectively exert the maximum therapeutic effect without causing deleterious side-effects on the host retina. Therapeutic effects of cell grafting can be evaluated by fundus imaging and OCT (gross retinal morphology), by electroretinography and visual evoked potentials (a measure of visual function), and by histopathology of the ocular tissue with retinal grafts in animals after they are terminated (projected: six months after blast injury). Postmortem analysis of the rabbit eyes includes histology, fluorescent immunohistochemistry and confocal microscopy with 3-D reconstruction of retinal tissue.

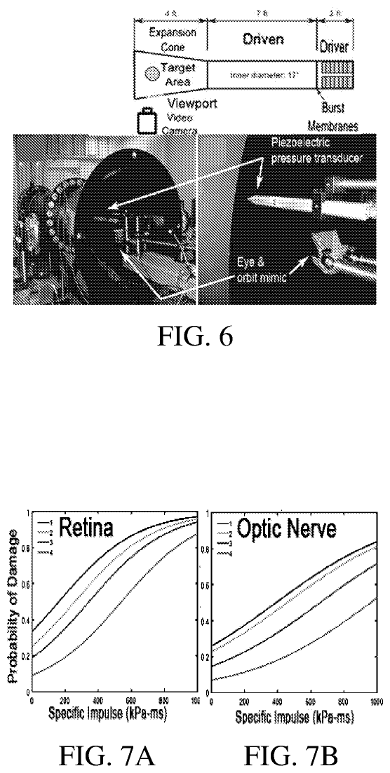

In this model, a large frame shock tube, as shown in FIG. 6, was used to produce a controllable primary blast wave without the addition of secondary or tertiary effects (Sherwood, D., et al., Anatomical manifestations of primary blast ocular trauma observed in a postmortem porcine model. Investigative Ophthalmology and Visual Sciences, 2014. 55(2): p. 1124-1132.). The “blasts” produced by this shock tube result in a range of peak static pressures from approximately 7 to 22 Pascals per square inch (psi) (48-152 kiloPascals, kPa), delivered in a Friedlander-like waveform with a positive pressure peak duration of 3.1 ms. Our data indicates that a survivable isolated primary blast is capable of producing acute retinal damage in rabbits (level 2-3, based on the cumulative injury scale (CIS) shown in Table 1.

TABLE 1
The Cumulative Injury Scale

| CIS | Severity of Injury |
|---|---|
| 0 | The eye is undamaged |
| 1 | The eye has some damage, but should heal fully on its own |
| 2 | The eye has damage that will require surgery to repair, |
| | leaving chronic pathology |
| 3 | The eye has damage that might be repairable with surgery, |
| | with severe visual loss |
| 4 | The eye is likely damaged beyond meaningful functional repair |

To predict the blast intensity for producing an injury of a given CIS, a “risk model” was developed based on the probability of the injuries produced over the range of blast intensities used. Ordinal logistic regression was applied to estimate the probability of achieving a given CIS score for each tissue component of the eye, for a given level of blast, including the retina and optic nerve, as illustrated in FIG. 7. To achieve an 80% probability of producing a retinal injury with CIS 3, a blast with a specific impulse of about 725 kPa per one millisecond (ms) (about 82 psi) would be required. Collectively, these data can be used as a guide to generate a cohort of rabbits with relatively uniform severity of retinal injury (and without optic nerve rupture, collectively, animals with “salvageable” vision problems) for statistical evaluation of the impact of cell therapies and retinal progenitor grafting on vision preservation. Short-distance axonal damage in neural retina is amenable to treatment with paracrine trophic factor support, while a ruptured optic nerve (e.g., in higher level CIS 3 injury in the shock tube) will lead to permanent vision loss that cannot be restored with current technologies.

The model includes about 96 specific pathogen-free (SPF)-grade New Zealand (NZ) pigmented brown rabbits, about 5 to 5.9 pounds each, supplied by RSI Robinson Services, Inc. Rabbits undergo an initial baseline structural and functional assessment using, for example, fundus imaging, OCT and ERG, VEP recording before receiving an ocular blast injury in the shock tube and are evaluated immediately after blast injury for structural and functional assessment. Rabbits rest in the ISR animal facility for 1 day and are moved to the UTHSCSA animal facility. Retinal organoids are dissociated to single cells and retinal progenitors are grafted into the rabbit eyes. About 4 rabbits may processed per day to maximize the quality of work, with about 2 hours spent on each animal.

Survival of human retinal progenitors in rabbit retina impacted by blast are evaluated. In addition, the ability to robustly deliver neuroprotection via paracrine secretion, while not causing damage to the host retina, will also be evaluated. Biomaterials generally promote cell survival in grafts. Epiretinal and subretinal grafts survive in mammalian retina but the cell integration dynamics may vary in rodents vs. a “large eye” model.

Cells from dissociated hPSC-3D retinal tissue are transplanted into the epiretinal and/or subretinal space of rabbits who have undergone controlled blast induced ocular injury resulting in damage to the retina and/or optic nerve. The neuroprotective effects are then measured by electroretinography (a functional assessment used to examine the light-sensitive cells of the eye, (rods and cones and their connecting ganglion cells in the retina) and visual evoked potentials (a functional assessment of the electrical stimulation of the occipital cortex in response to light outcomes). Histopathological analysis of the ocular tissue at selected time points after blast injury may also be performed.

The impact of subretinal and epiretinal grafting of hPSC-derived retinal progenitors with or without supportive biomaterial to ameliorate retinal degeneration after a blast injury are evaluated in rabbits. Preclinical and clinical testing of stem cells grafted into the ocular space showed therapeutic effect on degenerating retina. Biomaterials support the engraftment of retinal cells. Subretinal grafts can neuroprotect photoreceptors, while epiretinal grafts can support RGCs. Primary retinal progenitors can integrate structurally and functionally into the host retina.

Experimental procedures (methods) may include the following selection criteria for rabbits and pilot (P) experiments. The ex vivo pilot study on rabbit eyes showed that the grafts are easier to locate in a pigmented eye. F-1 NZ rabbits at about 5-5.9 pounds (2.5 kg), age about 3 months, were used to confirm the blast intensity (worked out on similar-size Dutch Belted rabbits) to achieve CIS 2-3 retinal injury, causing 50% drop in ERG amplitude and implicit time and/or VEP amplitude/latency. Rabbits are prescreened before the blast (to exclude ocular problems) and after (to confirm the expected CIS) by assays such as fundus imaging, OCT, ERG, and VEP. Rabbits should have CIS 2-3 retinal injuries. Grafts will include about 50,000 hPSC-retinal progenitors administered in both eyes, and also, into 3 NZ rabbit eyes without injuries. Eyes can then be assayed by, for example, OCT (at +1 day, +1 week, +1 month) to show that the cells were grafted. Retinal bulges may be observed. The rabbits may be examined at +1 month after grafting to determine (by IHC, for example) if the cells have survived. An immunosuppression regimen may be used if needed, including for example, prednisone (2 mg/kg, topical)+cyclosporine (5.0 mg/rabbit every 12 hours, orally) from −3 days − to +8 weeks after surgery.

Ocular Blast Injury: The shock tube (as described above) is used to generate CIS 2-3 retinal injury in rabbits (Table 1). Imaging (fundus photography, OCT) and electrophysiology (ERG, VEP) can be performed 1 day before the blast and 2 days after, as shown in Table 2.

Ocular grafting tool: Any appropriate grafting tool can be used for administering the graft. For example, a World Precision's UMP-3 pump for ocular delivery of cells, connected to Micro-4 controller, 100-μl Hamilton syringe and microcapillary [outer diameter 1.0 mm, with pulled polished opening]) system may be used. Ocular histology, fluorescent immunohistochemistry may be performed on lightly fixed frozen sections, as well as confocal immunofluorescent microscopy.

About 50,000 human retinal progenitors may be used in the graft, dissociated from hPSC-derived retinal tissue (organoids) with, for example, papain (Nasonkin, I., et al., Long-term, stable differentiation of human embryonic stem cell-derived neural precursors grafted into the adult mammalian neostriatum. Stem Cells, 2009. 27(10): p. 2414-26), in a volume of about 40-50 microliters. When grafting cells with a carrier or scaffold, such as a hydrogel like HYSTEM® biomaterial (gel), cells may be pre-mixed with the carrier or scaffold before each grafting. We will graft heat-inactivated (dead) retinal progenitors (with or without a carrier or scaffold) in “control” (counterpart) eyes, as shown in Table 2.

TABLE 2
Study Design for Cells and Bioprosthetic Patch
(Cells + Bioprothetic Material)
Histology, IHC

| OCT, ERG, | Subretinal ~50,000 | Control ~50,000 | Epiretinal ~50,000 | Control ~50,000 |
|---|---|---|---|---|
| VEP, etc. | Cells | Dead Cells | Cells | Dead Cells |
| 1 day | measure | measure | measure | measure |
| 1 week | measure | measure | measure | measure |
| 1 month | measure | measure | measure | measure |
| monthly | measure | measure | measure | measure |
| follow-ups | | | | |
| 6 months | measure/terminate | measure/terminate | measure/terminate | measure/terminate |

Initial analysis will be performed by in vivo evaluation of eyes (for example, OCT=retinal thickness, presence of grafts, ERG, VEP-functional vision tests), 1 day before the blast, and 2 days after the blast. Cells will then be grafted, and periodic measurements will be taken (Table 2). We expect that at day +1 after the blast, the animals will have at least a 50% decrement in ERG and VEP amplitude and/or latency, compared to the animals' baseline levels. The criterion level of functional recovery is a gain in the electrophysiological responses to at least about 25%, 30%, 40%, 50%, 60%, 70%, or 75% of baseline. When the animals reach this level of recovery, or at +6 months after the blast exposure without recovery, they will be euthanized. The eyes will be isolated and optic nerves for frozen IHC analysis will be taken to delineate the impact of the grafts on retinal preservation. Cell survival, graft retention, integration of human cells into the rabbit retina, changes in retinal thickness, level of glial and fibrotic scarring, retinal remodeling, cell death, retinal structure will be measured at 6 months after surgery. The experiments will be partially blinded. Rabbits will be assigned an ID number. Lab techs will not know whether the left or the right eye of each rabbit received live cells until the end of the experiments. This will maximize the objective assessment of the efficacy of neuroprotection. Lab techs will not know rabbit IDs when doing histology and IHC analysis until the end of the experiments.

Power analysis, statistical evaluation, sample size and controls: Okuno et al. found that the VEP amplitude variability (relative standard deviation [RSD], or the coefficient of variation) was ˜12%, while the latency was invariant (RSD ˜3%). This makes VEP a robust measure of visual function. Using Okuno's formula as the basis for a power calculation, we estimated that a minimum sample size of seven is needed for sufficient statistical power to detect a difference in means with a power of 80% (1-β, where β is the probability of a Type II error) and a p-value of 0.05. The sample size can be 10 eyes/cohort, which is sufficient for statistical evaluation of visual function changes (VEP) by ANOVA method and allows for some attrition in the group (e.g., due to failed grafts).

To increase cell survival, immunosuppression can be used. The impact on retinal thickness and VEP will be marginal. In addition, a carrier such as a hydrogel (e.g., HYSTEM® biomaterial) (BioTime, Inc.) with trophic factors (e.g., BDNF embedded into the gel, for slow release) can be used to increase the impact on retinal thickness and VEP.

Certain cell dosages grafted into the adult CNS will enable robust integration of cells. While pharmacologic-based therapy expectations (a dose-response relationship) are important, an aspect of this study is to find a cell dosage, which will not adversely impact the recipient retina (e.g., leaving a bulge with nonintegrated cells in the subretinal space or growing epiretinal membrane in epiretinal space).

Experimental procedures (Methods): Cell dosages of 10,000, 100,000, and 250,000 cells are tested for generation of grafts for integration into rabbit retina. In this case, the choice of three cell dosages may be focused at about 50,000 cells (e.g., 30,000; 45,000; 65,000 cells/graft). Experimental design is shown in Table 3; 10 rabbits may be assigned to each dose level.

TABLE 3
Study design for optimizing cell dosage for subretinal
vs. epiretinal, with or without carrier/scaffold.
Histology, IHC

| OCT, ERG, | ~10,000 | Control Dead | ~100,000 | Control ~10,000 | |
|---|---|---|---|---|---|
| VEP, etc. | Cells | Cells | Cells | Dead Cells | Control |
| 1 day | measure | measure | measure | measure | measure |
| 1 week | measure | measure | measure | measure | measure |
| 1 month | measure | measure | measure | measure | measure |
| monthly | measure | measure | measure | measure | measure |
| follow-ups | | | | | |
| 6 months | measure/terminate | measure/terminate | measure/terminate | measure/terminate | measure/terminate |

One eye of each animal will have the graft, and the other eye will be grafted with dead cells. The route of administration (subretinal or epiretinal, with or without biomaterial) are chosen based on initial results.

Paracrine factors produced by the grafts causing best neuroprotection may be identified, and then either overexpressing these molecules by grafts, or/and embedding these molecules in supportive biomaterial.

Provided herein is an assessment of the time after retinal blast injury for delivering retinal cell therapy to ameliorate vision loss in a rabbit model.

Retinal cells begin to die soon after the blast injury. RGCs and photoreceptors are most sensitive to cell death. However, a drop in initial visual acuity in the first days after ocular blast injury does not guarantee the vision is lost. Instead, this becomes clear in approximately 3-4 weeks. Vision declines gradually, caused by progressing cell death. During this time, at least some vision could be saved. Delayed analysis (by +2 weeks after blast injury) will be used to determine whether therapeutic intervention may still be able to protect retina. The results will be relevant to developing vision preservation approaches in wounded soldiers during triaging.

Cell preparation, grafting, randomization to reduce bias, cohort size, sample collection, handling, and power analysis are described above. In addition to the study design outlined in Table 4 and measuring retinal thickness and retinal cell preservation (as described), comparisons and quantification of cell death in rabbit retina, treated with grafts at +3 days vs. +2 weeks after the blast will be analyzed. Cleaved Caspase-3, γH2AX (early markers of apoptosis) and Tunnel staining (late marker of cell death) may be used. As a second readout, quantitating the presence of activated microglia (Iba-1 marker) as a measure of retinal remodeling and inflammation in controls and experimental cohorts may be performed. Also, the difference in synaptic bouton preservation in inner- and outer plexiform layers can be determined.

TABLE 4
Study design for testing the impact of a 2-week delay in retinal
cell grafting after the blast on retina and vision preservation.
Histology, IHC
20 rabbits may be treated at 3 days after blast
injury; 20 at 14 days after blast injury

| OCT, ERG, | Grafting on day | Control graft on day | Grafting on day | Control Graft on day |
|---|---|---|---|---|
| VEP, etc. | 3 after blast | 3 dead dells | 14 after blast | 14 Dead cells |
| 1 day | measure | measure | measure | measure |
| 1 week | measure | measure | measure | measure |
| 1 month | measure | measure | measure | measure |
| monthly | measure | measure | measure | measure |
| follow-ups | | | | |
| 6 months | measure/terminate | measure/terminate | measure/terminate | measure/terminate |

Cell therapies can be formulated for improved preservation of retinal thickness, lower apoptosis, retinal remodeling level and better preservation of synaptic layers in retina treated earlier (at day +3 after the blast).

### Example 6

hPSC-3D retinal tissue was transplanted into the subretinal space of wild type cat eyes following a pars plana vitrectomy (n=3 eyes). The hPSC-3D retinal tissue may be transplanted using any applicable method, such as that described in Seiler, M. J., et al., Functional and structural assessment of retinal sheet allograft transplantation in feline hereditary retinal degeneration. Vet Opthalmol, 2009. 12(3): p. 158-69, for example, incorporated by reference herein in its entirety. The eyes were examined clinically for adverse effects due to the presence of the subretinal graft by fundus examination and spectral domain optical coherence tomography (OCT) imaging. Five weeks following grafting, the cats were euthanized, and immunohistochemistry of retinal sections performed using human specific antibody (HNu, Ku80 and SC121) to assess the location, differentiation and lamination of the graft in the subretinal space. Oral prednisone at an anti-inflammatory dose was administered for the duration of the study.

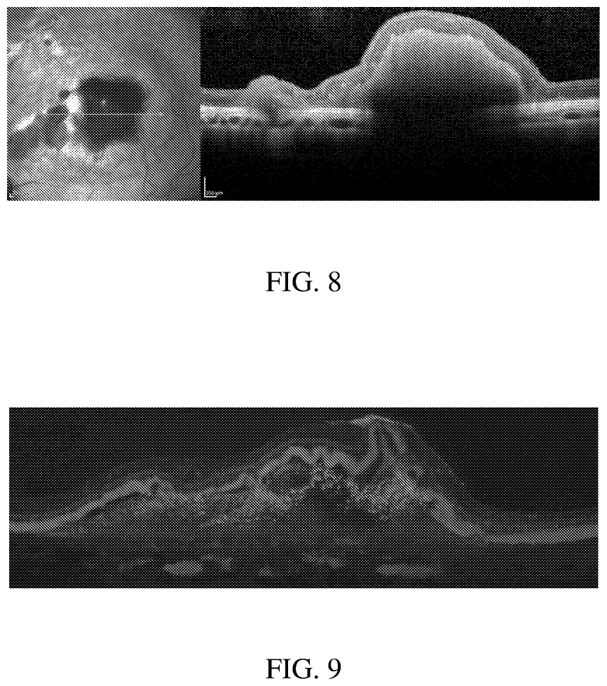

There was no gross retinal inflammation observed upon fundus examination. OCT imaging 3 weeks after grafting showed the presence of grafts in the correct location of the subretinal space, as shown in FIG. 8. Immunostaining of retinal cryosections with HNu and Ku80 antibodies also revealed the presence of the human derived retinal tissue grafts in the cat subretinal space, as shown in FIG. 9. The majority of cells in the graft had cytoplasmic staining instead of nuclear staining These results demonstrate that hESC derived retinal tissue can be successfully transplanted into the feline subretinal space without a severe inflammatory response.

### Example 7

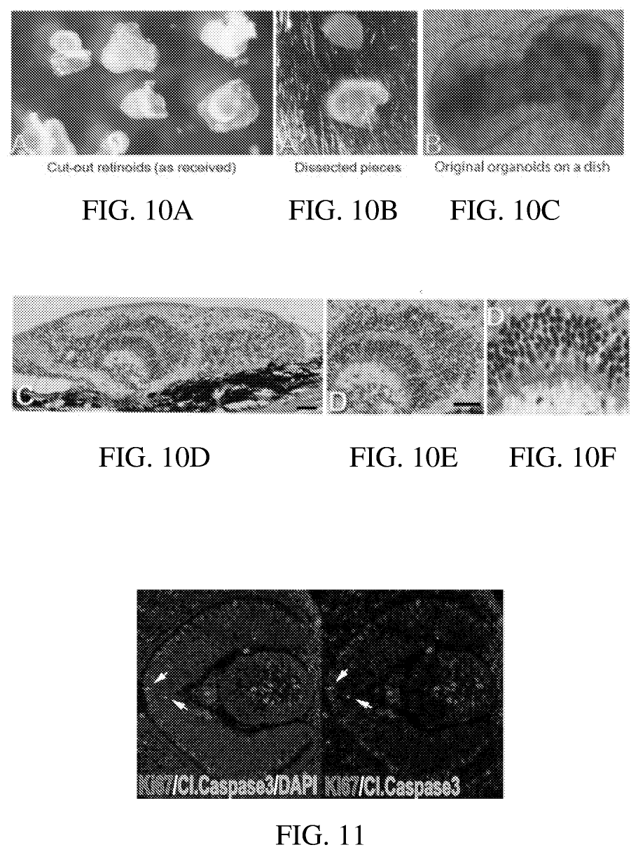

To demonstrate that implanted human embryonic stem cell-derived 3D retinal tissue (hESC-3D retinal tissue) has the ability to develop lamination within grafts, blind immunodeficient rats SD-Foxn1 Tg(S334ter)3 Lay (RDnude) rats were treated with hESC-3D retinal tissue delivered subretinally. FIG. 10A shows an image of hESC-3D retinal tissue (retinal organoids) dissected from a dish before transplantation. FIG. 10B shows an image of the dissected retinal organoids growing on a dish before transplantation. FIG. 10C is an additional image of a retinal organoids growing on a dish. After implantation and euthanization of the rats, histological analysis was performed on the subretinal space after 10 weeks from implantation. Lamination of the graft can be seen in FIG. 10D and FIG. 10E. In FIG. 10F, outer segment-like protrusions can be seen in the outer layer, immediately next to the rat RPE.

### Example 8

Overnight shipment of hESC-3D retinal tissue without impacting the viability of the retinal tissue in two different conditions (cold, in Hibernate-E medium, and at 37° C. in the original medium with or without BDNF) was demonstrated. Tissue was fixed on arrival and IHC with Cleaved Caspase-3 (an apoptosis marker) showed positive cells (FIG. 11, arrows), indicating that retinal tissue maintained viability after an overnight shipment in Hib-E at 4° C.

The feasibility of deriving 3D human retinal tissue carrying all retinal layers (PRs, 2^{nd} order neurons, retinal ganglion cells) and RPE from hESCs has been demonstrated (see for example International Patent Application Publication No. WO 2017/176810 incorporated herein by reference in its entirety). In addition, electrophysiology has been used to demonstrate that an increase in synaptogenesis coincides with an increase in electric activity within hESC-3D retinal tissue.

While only some neurons showed Na⁺ and K⁺ currents in 6-8 week-old hESC-3D retinal tissue, almost all tested retinal neurons in 12-15-week-old hESC-3D retinal tissue aggregates were electrically excitable and displayed robust Na⁺ and K⁺ currents.

### Example 9

World Precision Instrument's microcapillaries, with an outer diameter (OD) of 1.52 mm and inner diameter (ID) of 1.12 mm may be used. An immunosuppression regimen of systemic cyclosporine, from -7 days before grafting and onward, the technology of delivering hESC-3D retinal tissue into cat's subretinal space and imaging methods (e.g., Spectral OCT, RetCam at several different times, including immediately after grafting and immediately before terminating the animals), may also be used to deliver viable hESC-3D retinal tissue into the subretinal or epiretinal space of large eye animals.

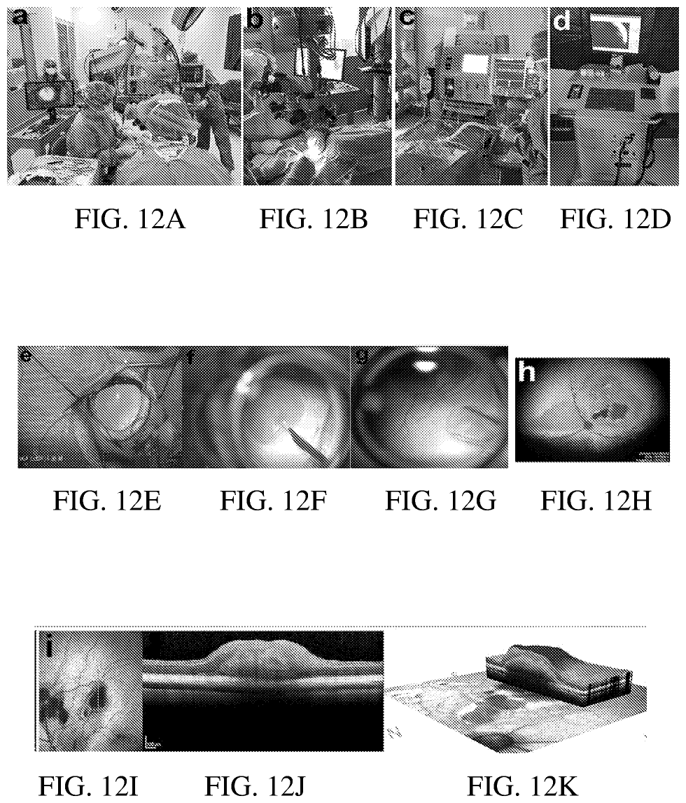

FIG. 12A through FIG. 12C show a surgical team transplanting hESC-3D retinal tissue into the subretinal space of a wild type cat. FIG. 12D shows the equipment for modulating ocular pressure and, RetCam equipment for imaging the grafts. FIG. 12E shows two ports inserted in a cat eye for intraocular surgery. FIG. 12F shows retinal detachment (a bleb), for grafting hESC-3D retinal tissue into the subretinal space. FIG. 12G shows a cannula for injecting hESC-3D retinal tissue. FIG. 12H shows hESC-3D retinal tissue in the subretinal space of a wild type cat, imaged with a RetCam. FIG. 12J shows a cross-sectional OCT image of hESC-3D retinal tissue placed in the subretinal space of a wild type cat, 5 weeks after grafting. FIG. 12K shows a 3D reconstruction of an OCT image to estimate the total size of the graft.

### Example 10

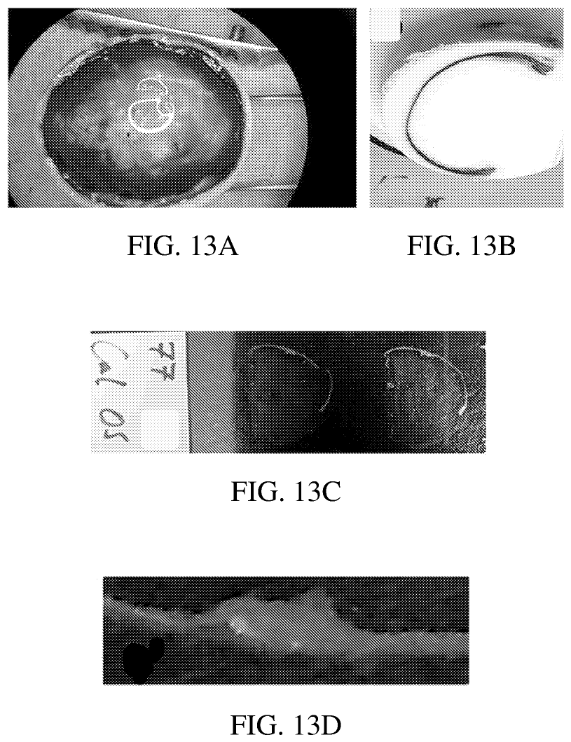

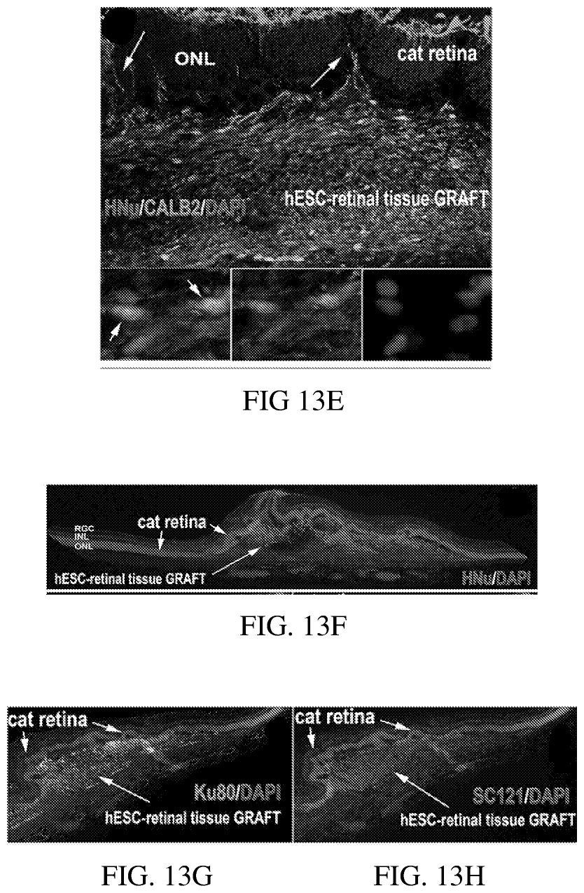

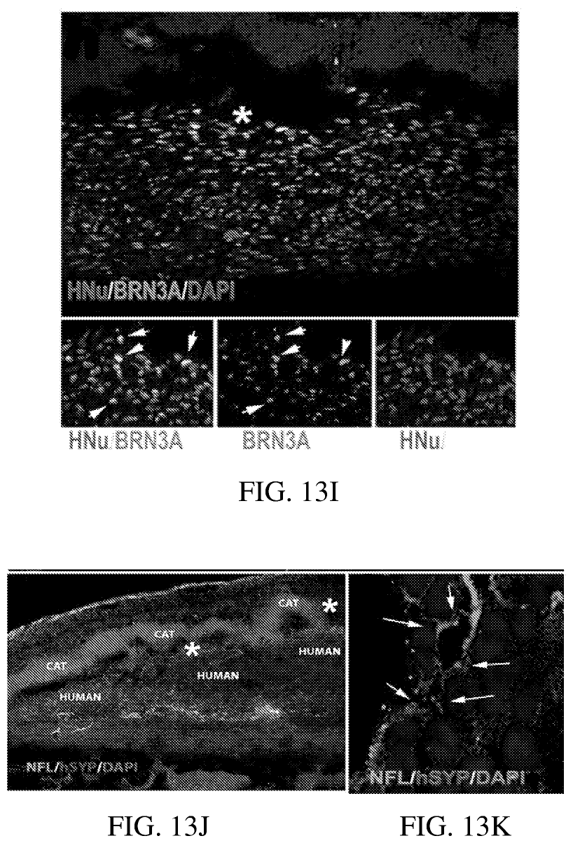

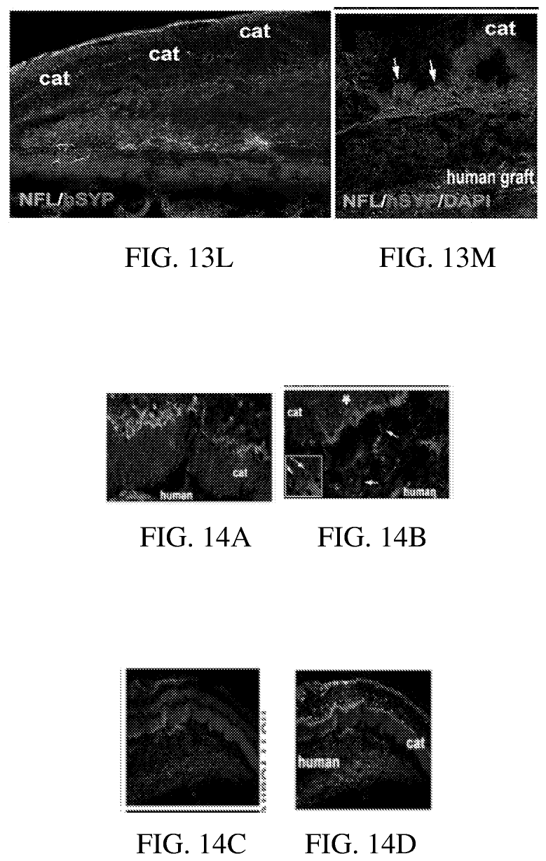

Immunohistochemical analysis of hESC-3D retinal tissue grafts in a wild type cat eye, 5 weeks after transplantation into the subretinal space demonstrated tumor-free structural and synaptic integration of hESC-3D retinal tissue into the retina of a large eye animal. Preservation of cat eye cups with grafts for frozen histology/IHC, confocal IHC with retina-specific, human-specific, synapse-specific antibodies was successfully performed. FIG. 13A shows a PFA-fixed, cryoprotected, OCT-saturated cat eye with subretinal graft, prepared for sectioning. FIG. 13B shows a cross-section of a cat eye frozen in OCT. FIG. 13C shows 16-μ-thick sections of a cat eye in OCT, which shows the graft as a bulge in the central retina. FIG. 13D shows a magnified image of the area of a frozen section showing preservation of hESC-3D retinal tissue grafts.

FIG. 13E shows IHC on a section of cat retina with hESC-3D retinal tissue graft, 5 weeks after grafting into the subretinal space. The graft shows the presence of many CALB2 (Calretinin)-positive neurons and the arrows point to CALB2[+] axons connecting human graft and cat's ONL. FIG. 13F through FIG. 13H show the hESC-3D retinal tissue graft in a cat's subretinal space, stained with HNu, Ku80 and SC121 human (but not cat)-specific antibodies, respectively. These results demonstrate that human tissue was in fact grafted into the correct location of the cat's subretinal space. FIG. 13I shows staining with BRN3A (marker of RGCs) and Human nuclei marker. The asterisks show the area with the markers in the main image, which are enlarged in the insets. These results indicate that cells within the graft are undergoing maturation towards RGCs. FIG. 13J through FIG. 13K show staining with antibodies specific to human (but not cat)-synaptophysin (hSYP) and axonal marker NFL (specific to both cat and human neurons) and shows the presence of puncta-like staining (arrows) which indicates potential synapses formed by human neurons, which are integrating into cat neurons. Human puncta-like staining was observed at the border between the cat ONL and the hESC-3D retinal tissue graft. This indicates potential initiation of synaptic connectivity. The pattern of distribution of the puncta-like staining (red) also demonstrates developing human synapses connecting to recipient retina.

Immunohistochemical (IHC) evidence of connectivity between the hESC-3D retinal tissue grafts in wild type cat's subretinal space was demonstrated 5 weeks after grafting. FIG. 14A and FIG. 14B show human (but not cat)-specific synaptophysin antibody hSYP (Red) and Calretinin (Green), which stains both cat and human neurons. hSYP stains human puncta in cat's ONL (arrows). FIG. 14C and FIG. 14D show lower magnification images, providing an overview on the large piece of cat retina with hESC-3D retinal tissue graft. hSYP staining originates in the graft and stains the graft, part of the ONL facing the graft but not the cat retina adjacent to the graft.

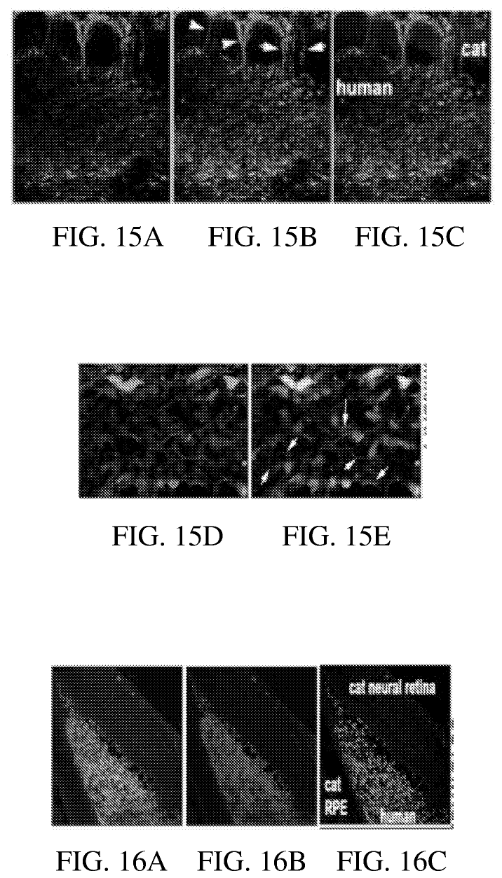

FIG. 15A through FIG. 15C show Calretinin[+] axons (arrows) connecting the cat INL and the Calretinin[+] human cells in the graft. Under higher magnification, these axons could be seen stretching from cat cells into human graft, and from human Calretinin[+] cells into cat INL. FIG. 15D and FIG. 15E show Calretinin[+] neurons in the graft, which appear mature and Calretinin[+] axons which were found throughout the grafts.

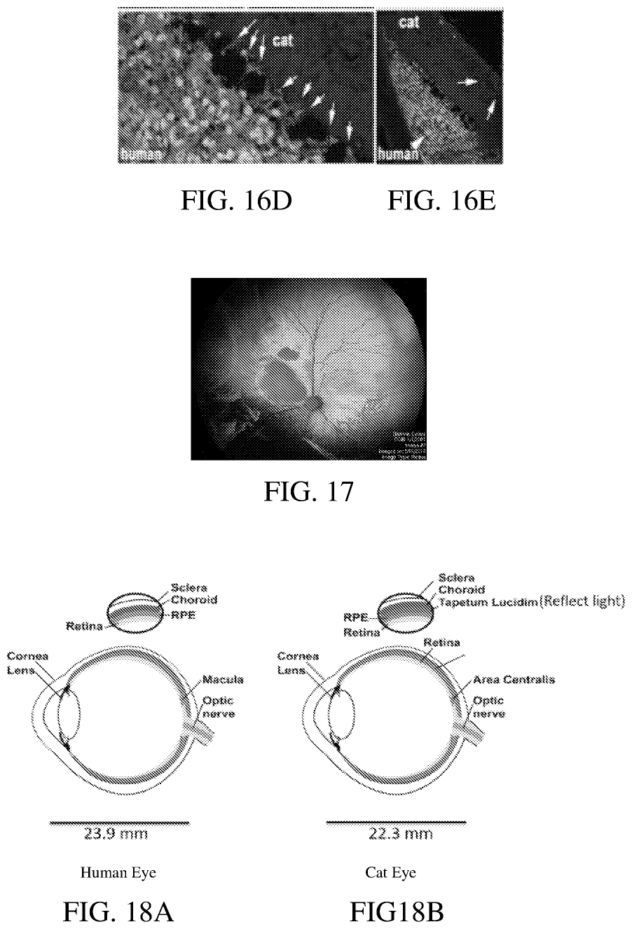

FIG. 16A through FIG. 16E show staining of the edge of the hESC-3D retinal tissue graft in the cat subretinal space. SC121 human cytoplasm-specific antibody (Red) and Ku80 human nuclei specific antibody (Green) stain human retinal graft but not cat retina. It can be seen from this image that there is graft to host connectivity. FIG. 16D shows the axons from hESC-3D retinal tissue graft wrap around (arrows) the cat PRs in the layer immediately next to the graft, while some SC121+ human axons can be seen crossing the cat's ONL (FIG. 16B, FIG. 16E, arrows).

These results indicate that the pattern of distribution of staining are indicative of synaptophysin stained synaptic connectivity resulting from the graft in addition to tumor free survival and maturation of the graft cells. No tumors developed in any of the cat subjects.

### Example 11

The mechanisms of synaptic connectivity based on histology and IHC and functional assessment (based on electrophysiology level of hESC-3D retinal tissue into the degenerating retina of at least two large eye genetic RD animal models will be further demonstrated. It has been demonstrated that hESC-3D retinal tissue taken at certain developmental time points of differentiation is able to integrate structurally and synaptically into the degenerating recipient retina and serve as a “biological patch” to restore vision in subjects with retinal degeneration, retinal disorders, and diseases, including advanced retinal degeneration. Furthermore, demonstrating positive therapeutic impact of hESC-3D retinal tissue grafting in a large eye animal model with retinal degeneration will enable further enhancements of a bioprosthetic retina consisting of many hESC-3D retinal tissue pieces on a bioprothetic material. Two large eye animal models (Pde6a[−/−] dog and Aipl1^{-/-} cat, and if needed, 2 additional large eye animal models (CngbI^{-/-} dog and Crx^{+/-} cat) may be used.

Full field ERG and mfERG will be performed to evaluate the function of degenerating retina and compare the changes in retinal function in the area around the graft (central retina) and the periphery in the subjects with grafts as well as in the control subjects. Retinas will be assayed using established MEA techniques for electrical activity from the individual RGC cells in the retinas with PR degeneration, specifically in the area above the grafts. Multielectrode array enables readout from many individual RGCs at once, thus obviating the need to use tedious patch-clamping on the individual RGCs, which will be less informative and may not indicate RGCs with the synaptic connectivity to the hESC-3D retinal tissue graft. The recording can be done in an oxygenated chamber for 1-2 hours which maintains the viability of retina, thus enabling the accurate readout. These assays enable analysis of the correlation of the synaptic connectivity on structural (histology/IHC with human Synaptophysin, human SC121 antibodies, and WGA-HRP transsynaptic tracer) and functional (electrophysiology) levels of the individual retinal cells. mfERG will allows for pinpointing the activity in the host retina (vs. individual cells) around the graft. Multielectrode array will enable demonstration that the graft works via cell replacement rather than (or in addition to) via neuroprotection mechanism/cell fusion.

Because the MEA recording takes about 1-2 hours and leads to gradual deterioration of retinal structure, hESC-3D retinal tissue may be grafted in both eyes of Pde6a dogs and Aipl-1 cats (6 animals=12 eyes/each model), which would allocate one eye for multielectrode array readout while the counterpart eye can be used for histology/IHC readout.

In vitro and in vivo hESC-3D retinal tissue expressing a transsynaptic tracer Wheat Germ Agglutinin-Horseradish Peroxidase (WGA-HRP) will be assayed and grafting of hESC-3D retinal tissue in both dog (Pde 6a[−/−]) and cat (Aipl-1[−/−] models of RD, 6 each) will be analyzed to evaluate both models for the ability to maintain the grafts and promote synaptic integration. Histology, IHC and xenograft-specific antibodies may also be used. In vitro electrophysiology (MEA) together with high resolution histology, immunohistochemistry, mfERG and VEP can be used to evaluate the outcomes of the grafting.

Provided herein are methods to determine the mechanisms of synaptic connectivity between the graft and the recipient degenerating retina grafted into 3 cohorts of animals (at the onset of RD, into partially degenerating retina with about 50% preservation of ONL thickness, and into retina with mostly/fully degenerated PRs). Both in vitro and in vivo electrophysiology, as well as visually guided behavior tests, can be used to delineate the extent of vision recovery in visually impaired subjects.

Grafting of bioprosthetic retina (a larger graft than the size of individual hESC-3D retinal tissue constructs or organoids) will also be performed. Bioprosthetic retina, where multiple pieces of hESC-3D retinal tissue pieces are mounted on a bioprothetic material or carrier or scaffold (for example, hydrogel based, for example, HYSTEM®)) will carry thousands of PRs (=biological pixels) and will enable restoration of visually-guided behavior. This bioprosthetic retina can also be customized to treat specific retinal diseases or disorders, such as macular degeneration, as the patch could be redesigned to carry mostly cones to rebuild macula, consisting mostly of cones.

Wheat germ agglutinin conjugated to horseradish peroxidase (WGA-HRP) can be used as a transsynaptic tracer. 3D retinal tissue may be derived from tracer[+] and tracer[−] hESCs, co-cultured for 2-3 months, and tested for HRP (using the HRP substrate DAB) in the tracer[−] retinal tissue, which would indicate transsynaptic tracer migration from tracer[+] retinal tissue. Co-cultures comprising tracer[+] human retinal tissue with dog and/or cat fetal retinas for 2-3 months can be used to assess synaptic connectivity by testing for either: 1) WGA-HRP migration, or 2) formation of chimeric human/nonhuman synapses. Feasibility was shown for the latter method with antibodies specific to human synaptophysin (hSYP) and human cytoplasm (SC121), though we will also attempt WGA-HRP as it would detect chimeric (human-nonhuman) synapses with higher sensitivity. Then tracer[+] retinal tissue constructs can be grafted into the subretinal space of young (4-5 week) Pde6a−/−dogs and 6 Aipl-1−/−cats (both eyes will receive the grafts). The animals can be imaged using RetCam and optical coherence tomography (OCT), the animals sacrificed at, for example, 6 months, samples stained for DAB, hSYP and SC121 to assess graft/host synaptic connectivity (one eye/animal) and the other eye tested by ex-vivo electrophysiology using multielectrode array (MEA).

Demonstrating both synaptic integration (by transsynaptic tracer and IHC), elongation of outer segments in PRs in grafts, as well as functional integration (finding RGC activity by MEA around/above the grafted area) by 6 months after grafting can be further demonstrated. A neuroprotective effect from young hESC-derived retinal organoid grafts may also be demonstrated. The observation of MEA signal (compared to retina 3-4 mm outside of the graft) may show that regeneration or slowing the progression of retinal degeneration is due to specific PR replacement mechanisms, rather than neuroprotection alone.

In one embodiment, hESCs expressing Wheat Germ Agglutinin-HRP genetic tracer under the control of Elongation Factor-1 alpha (EF-1α) promoter will be designed, hESC-3D retinal tissue derivation will be scaled up for production, the identity of hESCs (DNA fingerprinting) will be determined, karyotyping performed, transplantation of engineered hESC-3D retinal tissue into 6 Pde6a−/−dogs and 6 Aipl-1−/−cats (both eyes), OCT, full eye ERG, mfERG, MEA and VEP (using control Pde6a−/−dog and control Aipl-1−/−cat as control readout for retinal degeneration), wait 6 months, sacrifice the animals, isolation the eyes and the retinas with grafts, delineation of changes in RD retina function in the area above the graft (using patch clamping on individual RGCs, also MEA), then fixation of the tissue with graft, and delineation of the synaptic connectivity between the graft and the recipient and maturation of grafted hESC-3D retinal tissue using antibodies to retinal-specific immunophenotypes.

cGMP-grade hESCs may be used for derivation of hESC-3D retinal tissue. The dynamics of differentiation may be determined in several different lines of cGMP-hESCs from companies such as ES Cell International Pte. Ltd., for example. Cells from ES Cell International Pte. Ltd., have normal karyotype and are thoroughly characterized.

Synaptic connectivity within hESC-3D retinal tissue and between this tissue and recipient degenerating retina can be used to create a functional biological “retinal patch” to receive and transmit visual information from PRs of the graft to RGCs of the recipient retina. Rapid degeneration of the recipient retina may promote graft to host connectivity by bringing the graft and RGCs of the recipient retina into close proximity Collective evidence suggests that 6-12 week old hESC-3D retinal tissue will survive, differentiate, laminate and synaptically connect to recipient retina in dogs and cats with RD. Because the hESC-3D retinal tissue has a layer of RPE, the PR are well suited to survive and mature in grafts and develop outer segments.

A WGA-HRP trans-synaptic tracer may be used to demonstrate the synaptic connectivity between the graft and the host. WGA-HRP is expressed from a strong ubiquitous promoter, EFlalpha, and can be engineered by transducing EFlalpha-WGA-HRP construct in a custom-made lentiviral vector (GeneCopoeia, for example) into hESCs (from which hESC-3D retinal tissue are derived) and will be able to cross human/dog or human/cat synapses if the synaptic connectivity is established in 6 months. hESC-3D retinal tissue has been shown to (i) initiate synaptogenesis and axonogenesis by about the eighth week of development, and (ii) show signs of synaptic puncta in between the grafts and the recipient wild cat retina in less than two months after grafting. High-resolution confocal immunohistochemistry with hSYN antibody (specific to presynaptic part of human but not cat/dog synapse) and HNu (human nuclei) antibody may be used to demonstrate human synapses around the retinal neurons of the recipient. We can search for hSYN[+] boutons on the recipient neurons, which do not have HNu[+] nucleus and separately, and for SC121[−] axons with hSYN[+] boutons on them.

As an additional control, we may have an animal with a retinal degeneration mutation, which was not surgically manipulated, and will isolate and test the identical retinal area with MEA. The results can be compared to those where grafts were placed, which is not far from the optic nerve as our “landmark”.

Multielectrode array (MEA) may be performed on counterpart eyes as an ex-vivo electrophysiology experiment. We cannot use retinal tissue after MEA for histology, as it gradually loses its integrity. Therefore, we can perform MEA readouts from samples from about 6 dog and 6 cat eyes with grafts (after attempting to do mfERG and VEP in vivo, before the animals are terminated), while histology and IHC data are generated from the counterpart eyes (also 6 dog and 6 cat eyes with grafts). We can perform mfERG on both eyes of each animal before the animals are terminated and compare the signal from the retina around the graft with retina that has completely degenerated and PRs further away from the graft (as a negative control).

mTeSR1 media can be used and hPSCs cultured on Laminin-521 or Growth Factor Reduced (GFR) MATRIGEL or vitronectin. Custom made (by companies such as Genocopoeia) trans-synaptic reporters in a lentiviral vector can be transduced into hESCs, isolated using drug selection Puromycin for 2 weeks in 10 μM Rho-kinase inhibitor (ROCK), and colonies assayed for WGA-HRP expression, expanded and preserved in liquid nitrogen. Derivation of hESC-3D retina may be performed according to methods described herein. Eyes can be enucleated immediately after animals are terminated (MSU protocol or other protocol), immersed in ice-cold fresh 4% Paraformaldehyde/PBS pH7.6-8.0, anterior chamber removed, and eyecups fixed for an additional 15 min at 4 C.° for histology, IHC and preembedding. Eyecups can be cryopreserved in 20%-30% sucrose and snap-frozen in OCT/sucrose. We may also preserve the optic nerve and brain tissue of each animal (for tracing HRP[+] axons, to assess whether WGA-HRP is transported from the graft via RGCs of the recipient and along the RGC axons to the superior colliculus. Selected sections containing hESC-3D retinal tissue grafts can be stained with human-specific HNu and a-Synaptophysin antibodies for analysis of human grafts and human/rat, human/cat synapses. IHC may be performed using antibodies/protocols as described in Singh, R. K., et al., Characterization of Three-Dimensional Retinal Tissue Derived from Human Embryonic Stem Cells in Adherent Monolayer Cultures. Stem Cells Dev, 2015. 24(23): p. 2778-95, or another protocol.

hESC-3D retinal tissue grafts may be grafted into three cohorts of cyclosporine-immunosuppressed animals: (i) before the onset of retinal degeneration, (ii) when 1-2 photoreceptor layers are still present, and (iii) after advanced degeneration. We may derive retinal tissue grafts from dog induced pluripotent stem cells (iPSCs) and evaluate if their immune compatibility with the dog recipient can enhance survival and functional integration of the bioprosthetic retinal graft. RetCam and OCT may be used to monitor the grafts for 12 months. Functional assays may also be used to test retinal photosensitivity and visual function at 3, 6, 9 and 12 months, including electroretinography (ERG), multielectrode-array (MEA) recording, visual evoked potentials (VEP), pupillary light reflexes, and visually guided maze navigation. Animals may be sacrificed at 12 months after grafting to determine synaptic integration.

The mechanisms of synaptic connectivity between the graft and the recipient degenerating retina can be determined by performing grafting procedures described herein on the animals at the onset of RD, into partially degenerating retina with 50% preservation of ONL thickness, and/or into retina with mostly/fully degenerated PRs. Both in vitro and in vivo electrophysiology, as well as visually guided behavior tests, may be used to delineate the extent of vision recovery in visually impaired animals.

Spectral Domain-OCT and RetCam imaging can be performed by selecting the “good” grafts (example criteria may include: large transplants surviving in the central retina) by high resolution spectral domain (SD)-OCT at 2 weeks, then 3 weeks after surgery, and followed by additional SD-OCT scans (at 2, 3, 6, 9 and 12 months post grafting), until 1 year. Animals with excessive surgical trauma/ocular bleeding may be eliminated at the first RetCam and SD-OCT scan. Optokinetic testing on transplanted and sham surgery cats can be performed every 2 months, starting at 1-2 months after surgery. This test will evaluate whether cats can see moving stripes of a certain thickness (cycles/degree) and determine their spatial threshold. Each test may be performed twice either on the same or the next day. Videos can be evaluated by 2 independent investigators unaware of the animal's condition. Because of the variability of the test, group sizes of at least 6 may be used.

Multifocal (mf) ERG is a method which can compare PR function between different areas of an animal's retina and pinpoint the fine electrophysiological differences between the grafted area and the host retina with degenerated PR around the graft. Pupillary light reflexes can be performed for pupillometry recordings on all animals and sham surgeries at about +2 weeks, then +3 weeks after surgery, and then at about 2, 3, 6, 9 and 12 months post grafting, until about 1 year. VEP recordings may be performed on all animals and sham surgeries at +2 months, then +4, 6, 9, 12 months after surgery, until 1 year.

For histology/IHC, the eyes may be enucleated (immediately after terminating the animals) and fixed in ice-cold 4% paraformaldehyde (PFA) for 2 hours, then washed in ice-cold PBS 3 times (for about 30 min each), cryoprotected with sucrose (at a final concentration of about 30% in PBS), and sectioned on Cryostat to generate 12 μm cryosections through the eyes with grafts (selected by SD-OCT, for example). Histology can be performed with hematoxylin-eosin (H-E) or crestal violet (CV) on each 20^{th} section to identify sections with grafts. IHC can be performed with the antibodies specific for human (but not cat/dog) tissue (SC-121, Ku-80 or HNu, NF-70), diverse cat/dog/human-specific retinal cell types (rod and cone PRs, bipolar cells (e.g., CaBP5, PKCα, SCGN), amacrine (e.g. Calretinin), RGC markers (e.g. BRN3A, BRN3B) and synapses (SYP, SYT, BSN, PCLO, CTBP2, mGluR6, PSD-95 etc., including hSYP antibody, specific to human but not cat/dog Synaptophysin.

MEA recording may be performed by enucleating eyes immediately after animals are terminated, transporting the enucleated eyes to an oxygenated chamber, where retinal pieces with grafts may be carefully isolated and kept in the oxygenated chamber throughout the recording procedure. For immuno-EM, the following procedure may be followed: fix the eye in about 3% glutaraldehyde plus about 2% PFA immediately after enucleation, wash, embed in a gelatin-albumin mixture hardened with glutaraldehyde, produce vibratome sections, IHC with hSYP antibody using nonfluorescent approach (horseradish peroxidase as a secondary antibody), embed in resin and resection at the ultrathin level.

### Morphological and Functional Assessments of Bioprothetic Retinal Grafts

To assess the quality of the grafting procedure and whether the grafts induce photosensitivity in the degenerated retina, several morphological and functional assessments may be performed. Fundus imaging and optical coherence tomography may be performed periodically after grafting to monitor graft appearance and state of the retina. To probe for graft-induced photosensitivity, various behavioral and electrophysiological tests may be conducted just before grafting, and after grafting at 3, 6, 9 and 12 months, such as: 1) visually guided behaviors; 2) in vivo imaging of pupillary light reflexes; 3) in vivo electroretinography to assess retinal light responses; 4) visually evoked potential recording to assess transmission of retinal light responses to visual cortex; and 5) in vitro multielectrode-array recording to assess light responses of ganglion cells within the grafted retinal regions.

Wide-Field Color Fundus Imaging can be performed using a video fundus camera (RetCam II, Clarity Medical, for example) to record graft placement immediately post grafting and periodically to monitor graft appearance and record any inflammatory reactions. Monitoring can be performed in the conscious animal after pupillary dilation (Tropicamide) and application of a topical anesthetic (proparacaine).

Spectral Domain—Optical Coherence Tomography (OCT). A Spectralis instrument (by Heidelberg Engineering, for example) can be used to record scanning laser ophthalmoscope (cSLO) and retinal cross-sectional images (OCT) of the graft. This is performed under general anesthesia (induction propofol, intubation and maintenance on inhaled isoflurane delivered in O₂, for example) with the animals placed on a heating pad and maintained at 37° C. A lid speculum and conjunctival stay sutures can maintain the globe in primary gaze. Both infrared and autofluorescent cSLO imaging can be performed. High resolution line and volume scans may be used to record graft and host retina appearance; enhanced depth imaging (EDI) protocols can be used as needed. Repeat imaging may be performed and aligned to previous images using Heidelberg eye tracking software. This allows assessment of retinal morphology and retinal layer thicknesses of both the graft and overlying host retina. This will provide morphological data on the state of the retina and any associated abnormalities that might occur after the transplantation procedure, such as retinal detachment, edema, or thinning of the retina itself. FIG. 17 shows a RetCam image of an implanted retinal tissue bioprosthetic in a cat, imaged immediately post grafting into the subretinal space.

### Functional Assessment Protocols

Vision testing in dogs may be performed using a four-choice vision testing device previously utilized in retinal therapy experiments. The measures are percentage correct exit choice and exit times providing objective assessment of vision at scotopic, mesopic and photopic lighting levels. This can identify rod as well as cone mediated vision. Each eye may be tested in turn by occlusion of the other eye using an opaque contact lens. Vision testing in cats may consist of a number of different techniques.

These include assessment of the optokinetic reflex (OKR) using a custom-built optokinetic device and identifying a platform. OKR testing is a technique for vision assessment in cats. Utilization of the cat's behavior in tracking a moving object can also be used—i.e. tracking a laser pointer. Finally, a technique for assessing feline visual acuity, e.g., the ability to jump to a platform indicated by a visual stimulus, can be used. In this technique, cats are trained by rewarding them for identifying the indicated platform and providing negative reinforcement for choosing the incorrect platform.

In vivo pupillary light reflex (PLR) imaging may be used to determine whether the graft enhances retinal photosensitivity. Though the PLR is mediated almost entirely by intrinsically photosensitive retinal ganglion cells (ipRGCs), it is useful for assessing the functions of not only ipRGCs but also rod/cone circuits because ipRGCs respond to light both directly via their photopigment melanopsin, and indirectly via synaptic input from rods and cones. PLRs may be measured at a total of five timepoints as mentioned above. At each time point, the PLR imaging can be performed one day before in vivo ERG recordings are obtained from the same animals. All PLR imaging can be made at about the same time of day to minimize circadian variations.

The evening before each day of PLR imaging, animals may be dark-adapted overnight. In the following morning and under dim red light, the animals are anesthetized. After turning off the red light and allowing the animals to dark-adapt for 10 min, the RETImap system (Roland Consult) can be used to locate the graft in the grafted eye. This instrument is based on confocal laser scanning technology, by which an infrared laser is used to scan the retina without light-adapting it or producing a visual response; an image of the fundus is obtained with the cSLO and the grafted region identified. This same system can then be used to produce a visible wavelength of light that focally illuminates the graft-containing region in the grafted eye, and an eye tracker (SR Research EyeLink 1000 Plus, for example) can be used to image the non-grafted eye under infrared illumination to look for any consensual PLR. Four different intensities spanning at least 3 log units may be presented. As a control, the focal illumination can be delivered to the equivalent region in the non-grafted eye, and any consensual PLR imaged from the other eye. The pupil images captured by the eye tracker can be transmitted in real time to another computer via a frame grabber for offline analysis of pupil diameter. This measurement can utilize a LabVIEW-based image processing routine. For the cat, the horizontal diameter mid-pupil can be measured. The peak pupil constriction can be measured in each recording. For each stimulus intensity, the Mann-Whitney U test can be used to compare the peak constrictions caused by illumination of the grafted eyes with those caused by illuminating the non-grafted eyes. If the grafts indeed enable or enhance photosensitivity, we expect photostimulation of the grafted eyes to cause stronger pupil constriction than photostimulation of the non-grafted eyes. p In vivo electrophysiology can assess the ability of transplanted hESC derived retinal tissue bioprothetic implants to support light-evoked activity from transplanted retina. A battery of in vivo electrophysiological assessments can be used. ERG techniques can show if the graft is functional and improves retinal function. VEPs can show if there is transmission to visual cortex and, along with vision and PLR testing can assess the overall feasibility of the grafting techniques to improve retinal function. These measurements can be made in the intact animal and can be performed repeatedly over long follow-up periods. In animal models of inherited or induced retinal degeneration, the status of retinal function can be assessed by full-field or focal flash-evoked ERGs. After transplantation of the stem cell-derived suspensions or sheet implants, the light response of the grafts may be more effectively tested by focal rather than by full-field stimulation of the grafted tissue, especially if the host retina is degenerated. ffERG may also be performed. Focal and multifocal ERG testing can be carried out using the RETImap system. Identification of the grafted region can be done using RETImap as described above for PLR imaging, and this instrument can also be used to focus a light stimulus on that region to elicit a focal ERG. Each grafted region can be stimulated, and responses can be recorded and compared both to retinal regions that have not be grafted and also to the identical region of control (untreated) eyes. Alternatively, a multifocal ERG can be carried out. p When the grafts successfully form photoreceptors and form synaptic connections with the host retina, thereby providing light-activated neural activity, transmission of visual information can be achieved centrally over the optic tract. To demonstrate this, we can record over the visual cortical area (corresponding to area 17 in human eyes). This can be done simultaneously with the ERG recording by applying dermal or subdermal electrodes to the occipital area of the animal's head. The same stimuli that can be used to produce the ERG responses can also elicit VEPs, assuming there is functional integration of the grafts. Flash (non-patterned) and patterned (checkerboards or gratings) stimuli may be used, which can be generated by the RETImap system. p Animals can be dark-adapted overnight and prepped for recording under dim red light. Anesthesia, pupil dilation and globe positioning can be used as described herein for OCT. Initially, a scotopic testing protocol may be performed starting with luminances below normal rod threshold and with increasing stimuli strength to eventually record a mixed rod/cone response. Following the dark-adapted series, the animal can be light-adapted to a rod-suppressing background light and then a light-adapted luminance series performed. If VEP recordings are to be carried out, predicated on the presence of functional ERGs, then subdermal needle electrodes or gold cup electrodes (we can determine which electrode style produces the best recordings in these animals) can be placed along the midline over the occiput, near the inion. Placement of the recording electrodes near the inion has been shown to minimize ERG contamination of the VEP in dogs. If gold cup electrodes are to be used, the animal's scalp can be shaved over the midline of the skull and at least 1.5 cm laterally on either side, cleaned with 70% alcohol, and thoroughly air-dried. Conductive electrode paste can be applied to the selected recording location and the cup electrode firmly applied to the skin and held down with surgical tape. Needle electrodes may be inserted subdermally after the scalp cleaning step without the need to apply electrode paste.

The electrophysiological data can be analyzed in a quantitative fashion. For the ERG recordings, the a-wave and b-wave amplitudes and implicit times can be recorded and stored in a database. For VEP, two types of analysis may be used. For flash-VEP, the latency of the N1 and P1 peaks in the response waveform, and the amplitude of these peaks with respect to the signal baseline, can be measured. These parameters can be stored in the database. If we are able to record a pattern-reversal VEP, we can use the fast Fourier transform (FFT) referenced to the counterphase frequency of the stimulus pattern to analyze the waveforms and obtain the amplitude and phase components for the steady-state VEP response. These parameters can also be stored in a database so that all the electrophysiological parameters for each animal can be readily retrieved as a function of graft type, post-graft duration, and any other relevant treatment parameter. The primary endpoints of the analysis may be: (1) if visual recovery, defined as light-evoked activity in the ERG or VEP, occurs after retinal grafts; (2) the type of stem cell treatment (or lack thereof) that was administered to the animal; and (3) the time to first observation of the light-evoked responses.

In vitro multielectrode-array (MEA) recording: in vitro multielectrode-array (MEA) recording may be obtained from the grafted regions to directly assess the light response of retinal ganglion cells that are downstream from the grafted tissue. Because these in vitro recordings require euthanasia of the animals, they may be performed at the 12-month time point post-grafting, after the in vivo functional assessments have been completed. The evening before the day of MEA recording, animals may be dark-adapted overnight. The following morning and under dim red light, animals may be euthanized, and eyecups generated from both eyes by hemisecting the eyes, discarding the anterior halves, and removing the vitreous using forceps. The eyecups can be transferred to two capped 50 mL tubes containing Ames' medium, which and continuously gassed with 95% O₂ 5% CO₂ using a portable carbogen tank. The capped tubes may be kept inside a lightproof box while being transported.

The dog/cat/rabbit eyecups may be transferred to fresh Ames' medium and dark-adapted for another hour, during which time the grafted retina can be visually inspected under infrared viewers to locate the grafted region. After finding the graft, a blade can be used to cut out an approximately 2.5 mm×2.5 mm piece of the eyecup that includes the grafted tissue. This piece can be flattened onto a 60-electrode MEA with the ganglion cell side down, and action potentials recorded extracellularly from ganglion cells as previously described. In this preparation, the retina's attachment to the pigment epithelium, choroid and sclera will not be disturbed so that the grafted tissue can remain firmly attached, and the visual cycle responsible for regenerating photoexcitable photopigments well-preserved. An intensity series of 1 s-duration full-field light steps ranging from 8.6 log to 15.6 log photons cm⁻² s⁻¹ may be presented. MEA recordings may be made from either a region of the retina adjacent to the grafted region, or from the equivalent region in the non-grafted retina. For both sets of recordings (i.e. graft-containing retina and control retina), spikes can be sorted using Plexon Offline Sorter software, for example. Alternatively, photoresponse amplitude in each electrode can be easily quantified by calculating the variance in the raw recording during the 1-s light stimulus, and during the 1 s before stimulus onset, and the difference between the two variances used as the photoresponse amplitude.

To determine whether the ganglion cell photoresponses recorded from the grafted region are significantly greater than those from the control region, light-evoked changes in spike rate or in recording variance can be compared between the two regions using the Mann-Whitney U test, for example. For each stimulus intensity, statistical comparisons may be done separately for the following categories of light responses: 1) fast excitation at light onset; 2) fast inhibition at light onset; 3) fast excitation at light offset; 4) fast inhibition at light offset; and 5) sluggish excitation resembling the melanopsin-based photoresponse of ipRGCs. If the grafted tissue does enable or enhance the photosensitivity of rod/cone-driven retinal circuits, we may see that the rapid light responses (i.e. categories 1-4) are significantly stronger in the grafted region than in the control region. On the other hand, we may not see any difference in melanopsin-based photoresponses, as these may not be significantly affected by the grafts.

Behavioral methods for objective vision testing (an obstacle course designed for dogs and cats and optokinetic tracking for cats) may be carried out if we find improvement of vision in the eyes with grafts by mfERF VEP and pupillometry, for example.

Graft-host connectivity may be assessed using, for example, the following methods: 1) WGA-HRP transsynaptic tracer, expressed by the graft but not by the host cells; 2) IHC/immunoEM with human (but not cat/dog) cytoplasm-specific antibody SC121 and/or human (but not cat/dog)-specific synaptophysin antibody hSYP and/or postsynaptic marker in the area away from the human graft, in the recipient retina) or/and 3) IHC with hSYP+HNu antibodies and retinal cytoplasmic antibody (e.g., Recoverin, CALB2, or/and BRN3A/B), to show that human boutons are around the recipient (not human) neurons. Also, a nonviral retrograde tracer Cholera Toxin B (CtB) injected into the superior colliculus of a recipient animal to demonstrate connectivity may be used. We can inject the tracer 2 weeks before terminating the animals in the superior colliculus area and use IHC to locate CtB in the human graft.

Multiple pieces of hESC derived retinal tissue can be mounted on a bioprosthetic carrier or scaffold comprising, for example, a hydrogel (such as HYSTEM®) based electrospun sheet of biomaterial (˜3×5 mm), or electrospun silk or other biocompatible material suitable for implantation into the eye as described herein, to create a bioprothetic retinal patch. The bioprothetic retinal patch may be transplanted subretinally into a subject and the subject may be followed for 1 year using the above mentioned imaging, as well as full-field ERG or/and mfERG, and VEP. In addition, behavioral vision testing (an obstacle course for dogs and cats, and optokinetic tracking for cats) may be used.

A piece of bioprosthetic retina (3×5 mm, for example) can be grafted into the subretinal space of the model and grafts assessed in vivo with cross sectional retinal imaging by SD-OCT (also RetCam) at 1 week, then 2 weeks, then at 1, 2, 4, 6, 9, 12 months after grafting. Retinal function can be tested in vivo by mfERG (as well as full field ERG), and vision by behavioral testing (an obstacle course-dogs and cats, also optokinetic tracking for cats), VEP and pupillometry at 2, 4, 6, 9, 12 months after grafting. Following euthanasia, we may assess graft integration and connectivity with the host retina by histology and confocal IHC to show synaptogenesis and PR OS elongation. Preembedding immunoEM (synaptic connectivity graft to host) may also be used, and EM (to show PR outer segments in grafts).

Initially, bioprosthetic retina may be grafted into the subretinal space (central retina) of 3 or more animals. The animals may be immunosuppressed with prednisone+cyclosporine from about −7 days prior to surgery and ending at about 8 weeks after surgery. Bioprosthetic retina can be grafted in both eyes of each animal (n=3 grafts, total of 6 eyes) via transvitreal subretinal grafting approach We may have at least one animal with RD without grafts as an untreated control. The current method enables delivery of several pieces of hESC derived retinal tissue into a cat's subretinal space with precision, without causing major retinal detachment.

SD-OCT and RetCam imaging may be performed to assess the presence of grafted material at time point=0 (immediately after grafting, for the pilot cohort), and then at +1 week, and +2 weeks after grafting. This will demonstrate the delivery of the bioprosthetic graft as a sheet into the subretinal space as well as graft survival and will generate OCT and histological results. The grafts may be monitored for 1 year or more to generate functional data on PR function and vision improvement (mfERG, obstacle course, VEP), in addition to histological and IHC on hESC-3D retinal tissue maturation within the bioprosthetic retinal patch, as well as synaptic integration.

OCT may be used to monitor the grafts and mfERG to monitor changes in electrical activity in the grafted area versus about 3-4 mm outside of the graft. This may serve as a control set (e.g., same retina, different areas). By 6-12 months after grafting, most large eye RD models will have a completely degenerated PR layer, and the signal detectable by mfERG will be originating from the grafts.

TABLE 1
Example of Experimental design.

| | Experimental | Control type | Control type 2 | |
|---|---|---|---|---|
| | cohort | 1a, 1b | (mfERG, OCT) | Tests |
| Pilot 1 | At least 3 animals, | 1 animal: | Grafted eye-area around | OCT, mfERG, |
| | graft in both eyes | 1a: 1 eye no graft; | the graft vs. area 3-4 mm | VEP behavioral test |
| | | 1b: 2^{nd} eye sham-grafted | away from the graft | |
| Pilot 2 | At least 3 animals, | 1 animal: | Grafted eye-area around | OCT, mfERG, |
| | graft in both eyes | 1a: 1 eye no graft; | the graft vs. area 3-4 mm | VEP, behavioral test |
| | | 1b: 2^{nd} eye sham-grafted | away from the graft | |
| Main experiment | At least 3-4 animals | Counterpart eye as | No need to use the | OCT, mfERG, |
| Balanced | 1 eye grafted | control -balanced | same eye as control | VEP, behavioral test, |
| control design | | control design | | evaluate by 1-way ANOVA, |
| | | | | the Mann-Whitney U test |

Synaptic connectivity (graft to host) can be seen in animals with grafts by histology/IHC (between 3-5 months after grafting, which may be evaluated indirectly during the experiment as the function of the mfERG readout, and then directly after animals are terminated). Trans-synaptic tracing and in vivo methods (mfERG, pupillary light reflexes, functional vision tests such as VEP and visually guided behavior such as maze walk may be used. Tracing WGA-HRP from human grafts to recipient retinal neurons or/and IHC with SC121, hSYP, HNu and retinal cell type-specific antibodies or/and preembedding immnoEM are all methods to show functional graft to host synapses.

### Example 12

Retinal organoids (also known as retinal tissue grafts or retinal tissue bioprosthetic grafts or grafts) comprising hESC derived retinal tissue were transplanted, at about day 40 of differentiation, into the subretinal space of wild type cat eyes following a pars plana vitrectomy (n=7 eyes), as described herein, using a Borosilicate Glass cannula with an outer diameter of 1.52 mm and an inner diameter of 1.12 mm (from World Precision). In Group 1 (n=3), Prednisone was administered orally at an anti-inflammatory dose for the duration of the study (5 weeks). In Group 2 (n=4), Cyclosporine A was administered systemically starting 7 days before transplantation and then continuously for the duration of the study, in addition to Prednisone. The eyes were examined by fundoscopy and spectral domain optical coherence tomography (OCT) imaging for adverse effects due to the presence of the subretinal grafts or surgical procedure.

The cat retina, which is structurally similar to human retina, as shown in FIG. 18, provides a representative large eye animal model in which to demonstrate the efficacy of transplantation of hESC derived retinal tissue. In particular, cats have a cone rich region called the area centralis which is similar to the human macula.

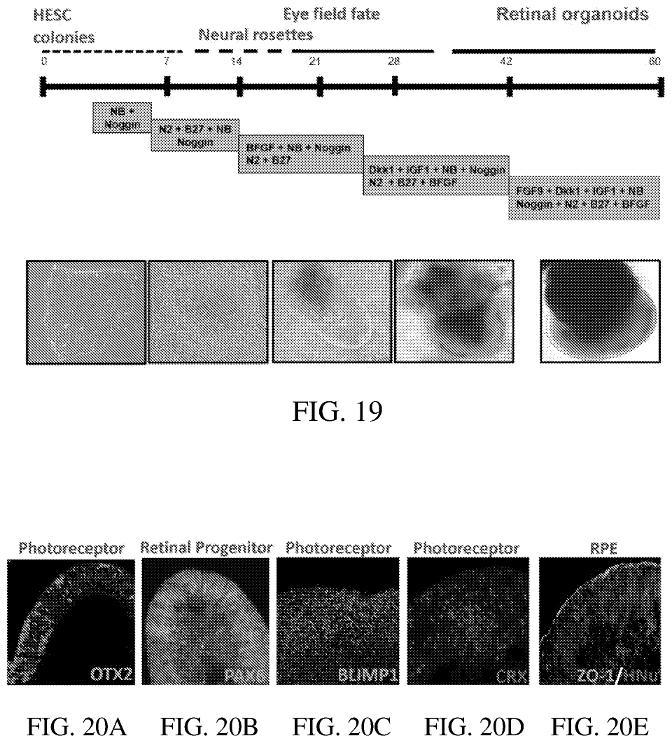

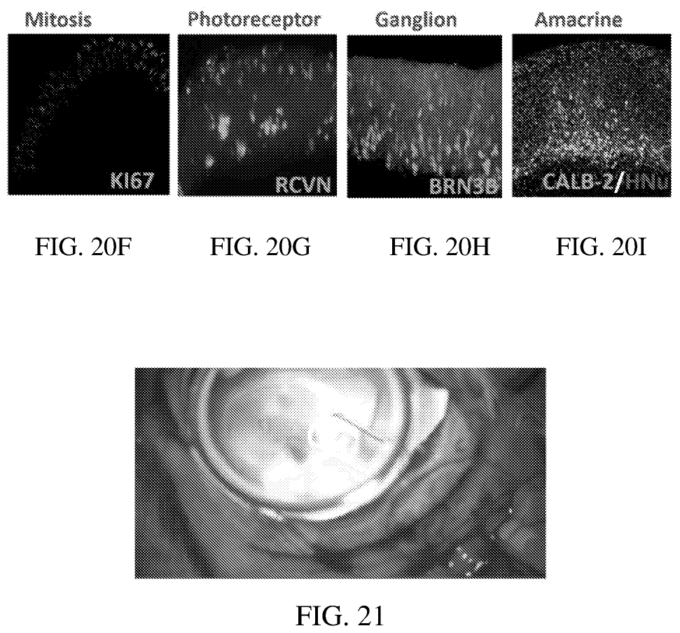

Retinal tissue constructs (organoids) were derived from human embryonic stem cell colonies using different morphogens, as described herein. An example of a timeline of retinal differentiation of retinal organoids is shown in FIG. 19. The expression of retinal progenitor markers and early photoreceptor markers in retinal organoids at 8 to 10 weeks was determined by immunostaining the retinal organoids using antibodies to retinal progenitor cell markers and early photoreceptor cell markers, as shown in FIG. 20A through FIG. 20I.

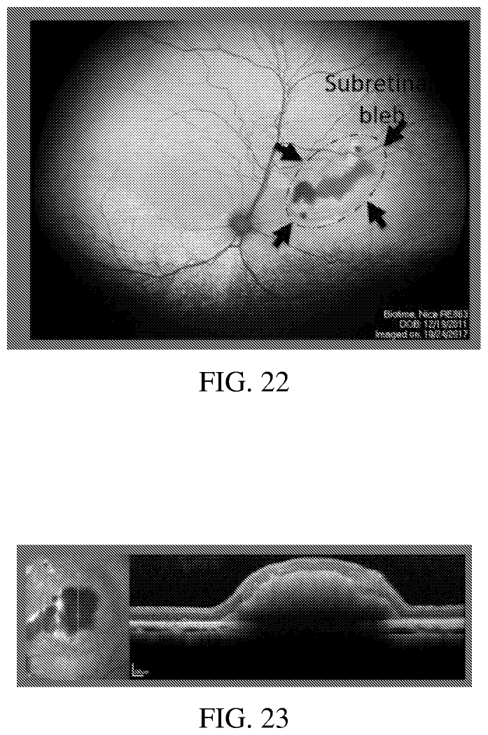

FIG. 21 shows an image of the transplantation of the retinal tissue graft into the subretinal space of a wild type cat eye following a pars plana vitrectomy using a glass cannula. A subretinal bleb was formed into which the retinal tissue graft is transplanted, as shown in FIG. 22. FIG. 23 shows the color fundus and OCT images taken at three weeks after grafting. The images indicate the presence and positioning of the graft in the subretinal space and show the absence of any severe adverse effects caused by the subretinal graft or surgical procedure.

Cats were euthanized 5 weeks following implantation of the graft. Immunohistochemistry (IHC) analysis of retinal sections was performed using human-specific antibodies (e.g., HNu, Ku80, SC121), axonal, synaptic, retinal cell type-specific markers and lymphocyte, microglia/macrophage markers.

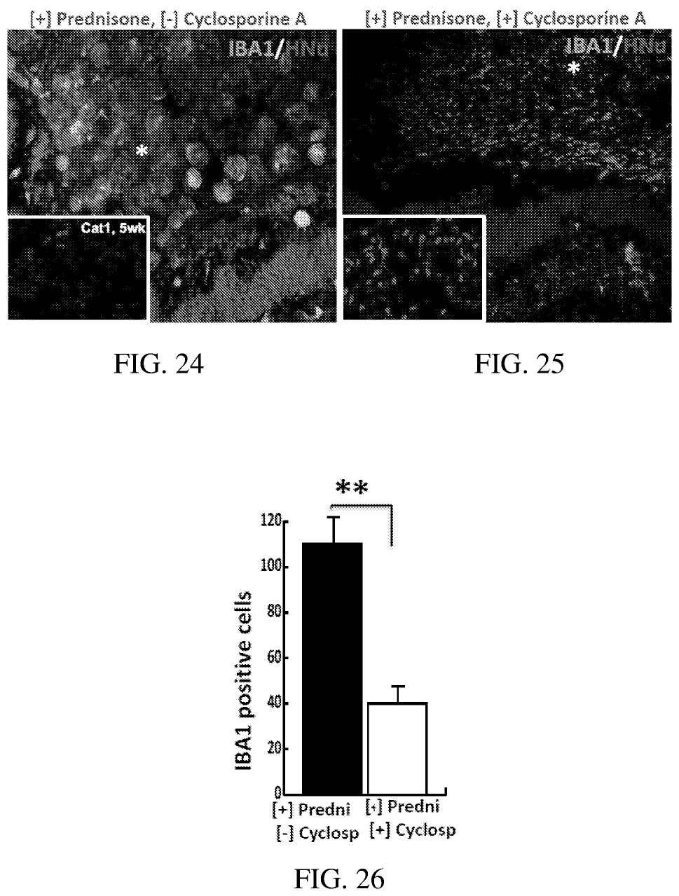

FIG. 24 shows an image of a retinal section from Group 1 (+Prednisone, −Cyclosporine A), stained using antibodies specific for microglia and macrophages. FIG. 25 shows an image of a retinal section taken from Group 2 (+Prednisone, +Cyclosporine A), also stained using antibodies specific for microglia and macrophages. As shown in FIG. 24 and FIG. 25, the addition of Cyclosporin A resulted in a decrease in the accumulation of microglia and macrophages (shown using IBA1 specific stain). In FIG. 25, the HNu human specific marker staining is well defined in the nuclei within the transplanted grafts, indicating that the cells of the graft survive at least 5 weeks post transplantation.

FIG. 26 shows a graph comparing the number of cells that are positive for microglia and macrophage cell markers in retinal sections for Group 1 (+Prednisone, −Cyclosporine A) and Group 2 (+Prednisone, +Cyclosporine A).

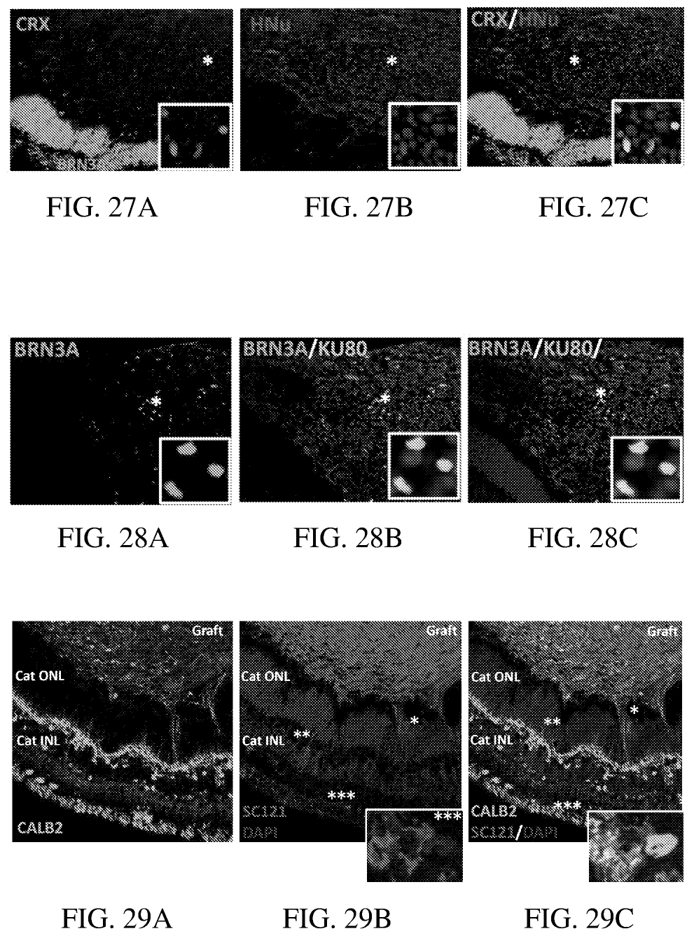

The positioning of the graft in the subretina of the cat can also be seen in FIG. 27A through FIG. 28C. FIG. 27A shows a cat retina section from Group 2 (+Prednisone, +Cyclosporine A) stained using antibodies specific for the photoreceptor marker, CRX. FIG. 27B shows a cat retinal section from Group 2 (+Prednisone, +Cyclosporine A) stained using human-specific antibodies, HNu. FIG. 27C shows a cat retinal section from Group 2 (+Prednisone, +Cyclosporine A) stained using antibodies to both CRX and HNu. As shown, the graft is positioned next to the cat's photoreceptor cells. In the magnified insert in FIG. 27C, cat photoreceptor cells and human cells are shown together. FIG. 28A shows a section of cat retina from Group 2 (+Prednisone, +Cyclosporine A) stained using antibodies specific for the retinal ganglion cell (RGC) marker, BRN3A. FIG. 28B shows a section of cat retina from Group 2 stained with both BRN3A and the human specific marker, KU80. The cell nuclei are also stained in FIG. 28C.

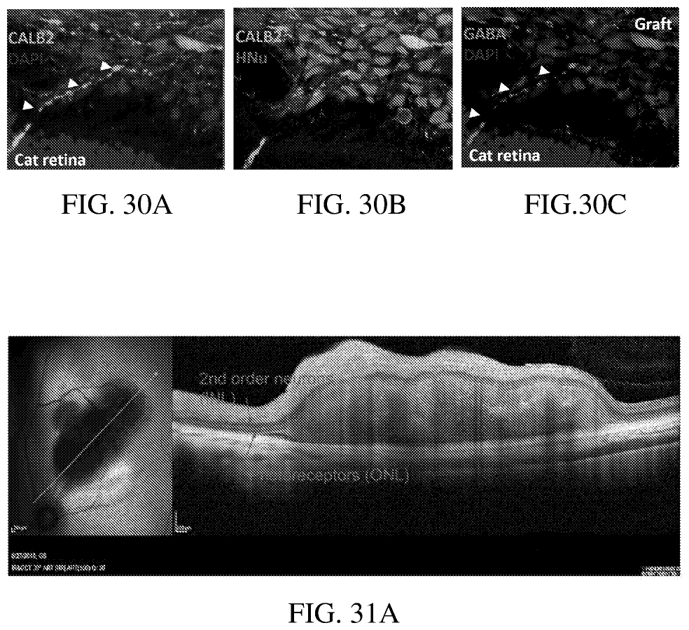

Axonal outgrowth of grafted hESC-retinal tissue was shown connecting to the recipient retina at about 5 weeks after transplantation. FIG. 29A shows a cat retinal section stained using antibodies specific for the Calretinin marker, CALB2, which is expressed in neurons, including retina. Cells positive for the expression of CALB2 can be seen stained in FIG. 29A, FIG. 29B and FIG. 29C. IHC analysis demonstrates that several axons emanating from the grafted hESC-derived retinal tissue grafts are positive for the expression of calretinin. FIG. 29B shows IHC staining for the marker, SC121. Antibodies to SC121 are specific for human cell cytoplasm. Thus, the position of the axonal outgrowth of the graft can be seen in relation to the recipient (cat) retinal ganglion cells, stained using DAPI. The IHC analysis shown in FIG. 29C demonstrates that at least 5 weeks after graft transplantation, axons from the graft have expanded and integrated into the outer nuclear layer (ONL), into the inner nuclear layer (INL) and even into the ganglion cell layer (GCL) of the recipient's eye.

In addition, ICH analysis was used to demonstrate that the transplanted human retinal tissue graft (positive for calretinin), which is capable of integrating into the recipient retina, was also GABAergic, as shown in FIG. 30A through FIG. 30C. FIG. 30A shows the axons of the retinal graft (stained using antibodies specific for the CALB2 marker) extending towards the cat retina. FIG. 30B shows the retinal graft stained with antibodies specific for the human cell markers, HNu and CALB2, thereby delineating the graft from the cat retina. GABA positive staining of the graft axons, shown in FIG. 30C, further indicate that the axons from the implanted tissue integrating into the recipient retina are differentiating towards a neuronal fate. These results demonstrate structural and functional integration of implanted hESC tissue and recipient retina.

The ICH analysis also indicated in vivo tumor free survival of the transplanted hESC-derived tissue for at least 5 weeks.

### Example 13

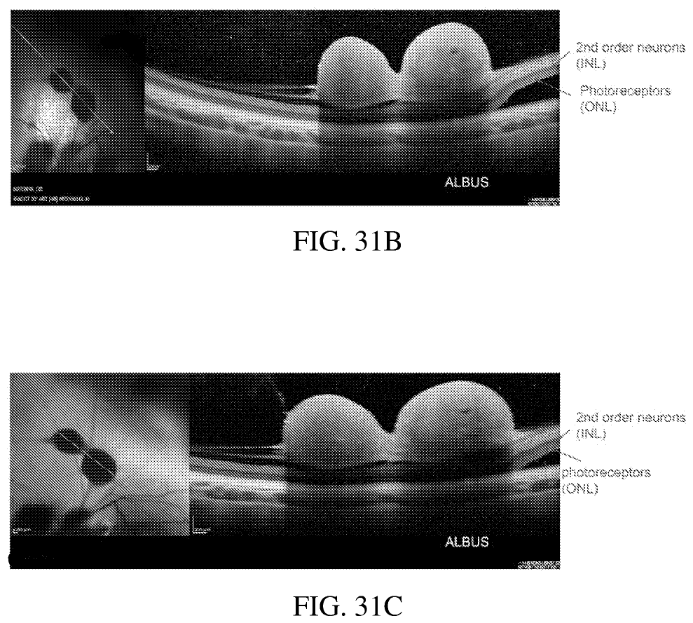

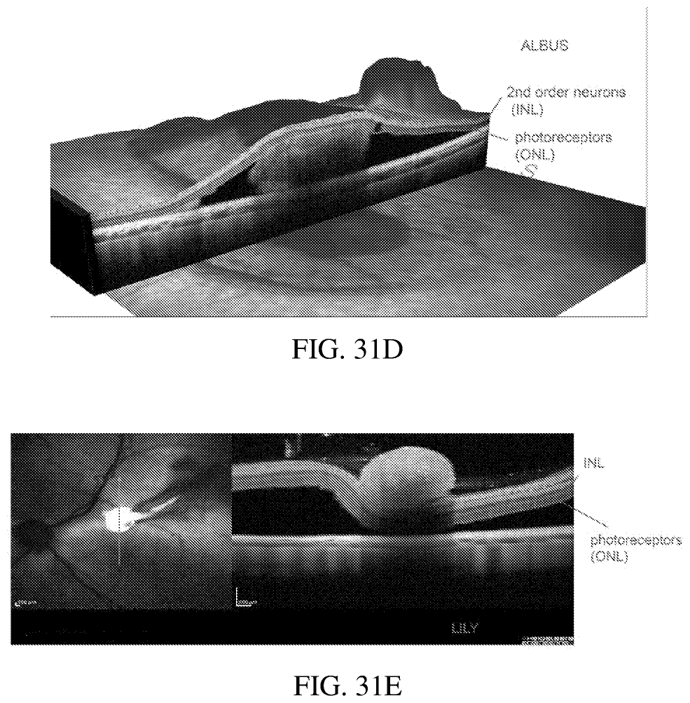

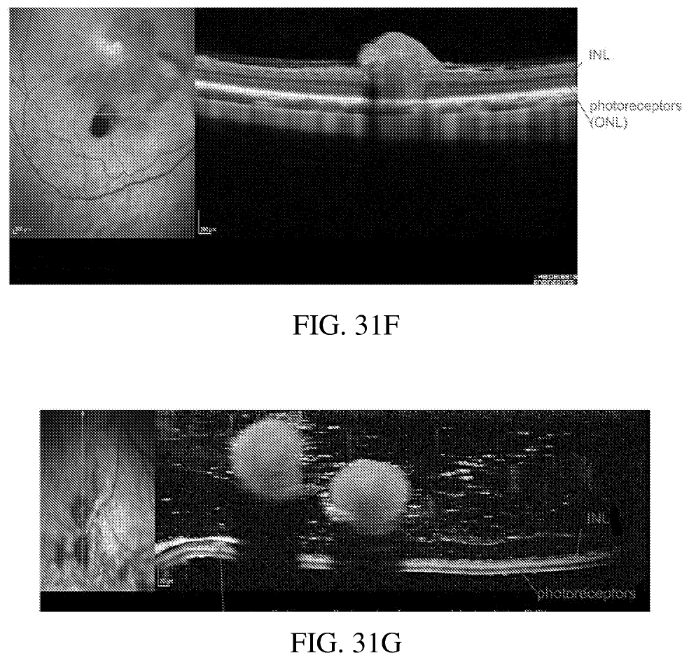

Retinal organoids comprising hESC derived retinal tissue were transplanted, at about day 40 of differentiation, into the subretinal or epiretinal space of CRX-mutant cat eyes with retinal degeneration following a pars plana vitrectomy, as described herein, using a Borosilicate Glass cannula with an outer diameter of 1.52 mm and an inner diameter of 1.12 mm (from World Precision). Cyclosporin A was administered systemically starting 7 days before transplantation and then continuously for the duration of the study, in addition to Prednisone, which was administered orally at an anti-inflammatory dose. OCT images were taken 3 months after implantation of the grafts. FIG. 31A through FIG. 31G show OCT images from two subjects and demonstrate that hESC derived retinal tissue grafts transplanted in the subretinal or epiretinal space of a large eye animal model with retinal degeneration (CRX-mutant cats) are capable of surviving for at least 3 weeks after transplantation.

### Example 14

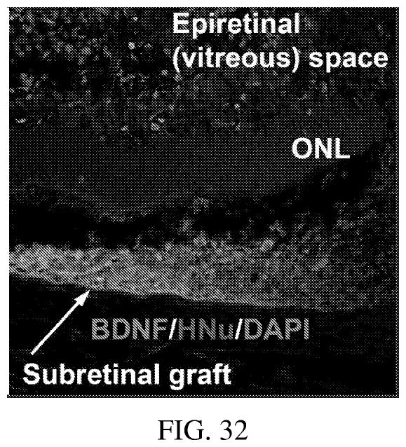

Turning to FIG. 32, BDNF expression was seen in hESC derived retinal organoids grafted into the subretinal space of a wild type cat, 5 weeks after grafting. As shown, most cells are BDNF+. BDNF is one of the key neurotrophins that supports the function of degenerating or damaged neurons. Higher BDNF levels can protect retina from retinal degeneration caused by disease or injuries. These results indicate that hESC derived retinal tissue grafts can provide neurotrophic support to damaged or degenerating retinal tissue after implantation into the ocular space of a subject's eye.

From the description herein, it will be appreciated that that the present disclosure encompasses multiple embodiments which include, but are not limited to, the following:

A method of one or more of, treating retinal damage, slowing the progression of retinal damage, preventing retinal damage, replacing retinal tissue and restoring damaged retinal tissue, the method comprising: administering hESC-derived retinal tissue to a subject. A method of one or more of, slowing the progression of retinal degenerative disease, slowing the progression of retinal degenerative disease after traumatic injury, slowing the progression of age related macular degeneration (AMD), stabilizing retinal disease, preventing retinal degenerative disease, preventing retinal degenerative disease after traumatic injury, preventing AMD, restoring retinal pigment epithelium (RPE), photoreceptor cells (PRCs) and retinal ganglion cells (RGCs) lost from disease, injury or genetic abnormalities, increasing RPE, PRCs and RCGs or treating RPE, PRCs and RCG defects, the method comprising: administering hESC-derived retinal tissue to a subject.

The method of any previous embodiment, wherein retinal damage is caused by one or more of, blast exposure, genetic disorder, retinal disease, and retinal injury. The method of any previous embodiment, wherein retinal disease comprises a retinal degenerative disease. The method of any previous embodiment, wherein retinal damage is caused by one or more of, Age-Related Macular Degeneration (AMD), retinitis pigmentosa (RP), and Leber's Congenital Amaurosis (LCA).

The method of any previous embodiment, wherein the hESC-derived retinal tissue comprises retinal pigmented epithelial (RPE) cells, retinal ganglion cells (RGCs), and photoreceptor (PR) cells. The method of any previous embodiment, wherein the RPE, RGC and PR cells are configured to form a central core of retinal pigmented epithelial (RPE) cells, and, moving radially outward from the RPE cell core, a layer of retinal ganglion cells (RGCs), a layer of second-order retinal neurons (corresponding to the inner nuclear layer of the mature retina), a layer of photoreceptor (PR) cells, and an outer layer of RPE cells. The method of any previous embodiment, wherein each of the layers comprise differentiated cells characteristic of the cells within the corresponding layer of human retinal tissue. The method of any previous embodiment, wherein the layers comprise substantially fully differentiated cells.

The method of any previous embodiment, wherein the hESC-derived retinal tissue further comprises a biocompatible scaffold to form a biological retinal prosthetic device. The method of any previous embodiment, wherein the biological retinal prosthetic device comprises between about 10,000 and 100,000 photoreceptors. The method of any previous embodiment, wherein the hESC-derived retinal tissue is capable of delivering trophic and neurotrophic factors and mitogens. The method of any previous embodiment, wherein the trophic and neurotrophic factors and mitogens comprise one or more of, brain-derived neurotrophic factor (BDNF), glial-derived neurotrophic factor (GDNF), neurotrophin-4 (NT4), Nerve Growth Factor-beta (βNGF) and pro-survival mitogen basic fibroblast growth factor (bFGF=FGF-2).

The method of any previous embodiment, wherein administration of the hESC-derived retinal tissue results in preservation of retinal layer thickness for between about 1 to about 3 months. The method of any previous embodiment, further comprising administration of immunosuppressive drugs. The method of any previous embodiment, wherein the immunosuppressive drugs are administered before, during and/or after the administration.

The method of any previous embodiment, wherein the method further comprises modulating the ocular pressure. The method of any previous embodiment, wherein the modulating the ocular pressure is before, during and/or after the administration of the retinal tissue.

The method of any previous embodiment, wherein the tissue is administered with an ocular grafting tool. The method of any previous embodiment, wherein the hESC-derived retinal tissue is administered subretinally or epiretinally. The method of any previous embodiment, wherein administration of the hESC-derived retinal tissue results in tumor-free integration of the hESC-derived retinal tissue and retinal tissue of the subject.

The method of any previous embodiment, wherein integration occurs between about 4 to 5 weeks after administration. The method of any previous embodiment, wherein administering does not cause retinal inflammation. The retinal tissue graft of any previous embodiment, wherein after administering, the retinal tissue develops lamination.

The method of any previous embodiment, wherein after administering, the retinal tissue neurons show signs of Na⁺ and/or K⁺ currents. The method of any previous embodiment, further comprising, demonstrating connectivity between the retinal tissue and existing tissue. The method of any previous embodiment, wherein the connection is demonstrated by one or more of: WGA-HRP trans-synaptic tracer, histology, IHC or electrophysiology. The method of any previous embodiment, further comprising measuring a level of functional recovery. The method of any previous embodiment, wherein a level of functional recovery comprises a gain in the electrophysiological responses that is at least 75% of a baseline.

Retinal tissue graft for transplantation into an eye of a subject, comprising: retinal pigmented epithelial (RPE) cells, retinal ganglion cells (RGCs), second-order retinal neurons, and photoreceptor (PR) cells, wherein the RPE, RGC and PR cells are configured to form a central core. The retinal tissue grafts of any previous embodiment, wherein there are from between about 10,000 and 100,000 photoreceptors. The retinal tissue graft of any previous embodiment, wherein the second-order retinal neurons correspond to the inner nuclear layer of the mature retina. The retinal tissue graft of any previous embodiment, wherein the cells are arranged such that moving radially outward from the core, the retinal tissue comprises a layer of retinal ganglion cells (RGCs), a layer of second-order retinal neurons, a layer of photoreceptor (PR) cells, and an outer layer of RPE cells. The retinal tissue graft of any previous embodiment, wherein the graft comprises from between 5,000 to about 250,000 cells. The retinal tissue graft of any previous embodiment, wherein the graft is transplanted into the subretinal space or epiretinal space.

The retinal tissue graft of any previous embodiment, wherein an increase in synaptogenesis coincides with increase in electric activity. The retinal tissue graft of any previous embodiment, wherein after transplantation neurons connect the graft to existing tissue. The retinal tissue graft of any previous embodiment, wherein the neurons are CALB2-positive. The retinal tissue of any previous embodiment, wherein connectivity is demonstrated by WGA-HRP trans-synaptic tracer. The retinal tissue graft of any previous embodiment, wherein after transplantation axons connect the graft to existing tissue. The retinal tissue of any previous embodiment, wherein the axons are CALB2-positive. The retinal tissue graft of any previous embodiment, wherein after transplantation, cells of the graft mature toward RGCs.

The retinal tissue graft of any previous embodiment, wherein after transplantation the graft forms synapses with existing neurons. The retinal tissue graft of any previous embodiment, wherein after transplantation the graft and existing tissue form connections. The retinal tissue of any previous embodiment, wherein the connections form within one day to about 5 weeks after transplantation. The retinal tissue graft of any previous embodiment, wherein after transplantation the graft forms axons which cross the existing tissue ONL.

The retinal tissue graft of any previous embodiment, wherein the graft produces paracrine factors. The retinal tissue graft of any previous embodiment, wherein the paracrine factors are produced prior and/or after to administration. The retinal tissue graft of any previous embodiment, wherein the graft produces neurotrophic factors. The retinal tissue graft of any previous embodiment, wherein the graft produces neurotrophic factors prior to or after administration. The retinal tissue of any previous embodiment, wherein the neurotrophic factors comprise one or more of, BDNS, GDNF, bNGF, NT4, or bFGF.

The retinal tissue graft of any previous embodiment, wherein after transplantation, the level of functional recovery is measured as a gain in the electrophysiological responses. The retinal tissue graft of any previous embodiment, wherein the level of functional recovery is measured as a gain in the electrophysiological responses to at least 10% of baseline. The retinal tissue graft of any previous embodiment, wherein after transplantation axons of the graft integrate into existing tissue.

In the claims, reference to an element in the singular is not intended to mean “one and only one” unless explicitly so stated, but rather “one or more.” All structural, chemical, and functional equivalents to the elements of the disclosed embodiments that are known to those of ordinary skill in the art are expressly incorporated herein by reference and are intended to be encompassed by the present claims. Furthermore, no element, component, or method step in the present disclosure is intended to be dedicated to the public regardless of whether the element, component, or method step is explicitly recited in the claims. No claim element herein is to be construed as a “means plus function” element unless the element is expressly recited using the phrase “means for”. No claim element herein is to be construed as a “step plus function” element unless the element is expressly recited using the phrase “step for”.

What is claimed is:

1. A method of one or more of, treating retinal damage, slowing the progression of retinal damage, preventing retinal damage, replacing retinal tissue and restoring damaged retinal tissue, the method comprising: administering a hESC-derived retinal tissue graft to a subject.

2. A method of one or more of, slowing the progression of retinal degenerative disease, slowing the progression of retinal degenerative disease after traumatic injury, slowing the progression of age related macular degeneration (AMD), slowing the progression of genetic retinal diseases, stabilizing retinal disease, preventing retinal degenerative disease, preventing retinal degenerative disease after traumatic injury, improving vision or visual perception, preventing AMD, restoring retinal pigment epithelium (RPE), photoreceptor cells (PRCs) and retinal ganglion cells (RGCs) lost from disease, injury or genetic abnormalities, increasing RPE, PRCs and RCGs or treating RPE, PRCs and RCG defects, the method comprising: administering a hESC-derived retinal tissue graft to a subject.

3. The method of claim 1, wherein retinal damage is caused by one or more of, blast exposure, genetic disorder, retinal disease, and retinal injury.

4. The method of claim 3, wherein retinal disease comprises a retinal degenerative disease.

5. The method of claim 1, wherein retinal damage is caused by one or more of, Age-Related Macular Degeneration (AMD), retinitis pigmentosa (RP), and Leber's Congenital Amaurosis (LCA).

6. The method of claim 1 or 2, wherein the hESC derived retinal tissue comprises retinal pigmented epithelial (RPE) cells, retinal ganglion cells (RGCs), and photoreceptor (PR) cells.

7. The method of claim 6, wherein the RPE, RGC and PR cells are configured such that there is a central layer of retinal pigmented epithelial (RPE) cells, and, moving radially outward from the RPE cell layer, a layer of retinal ganglion cells (RGCs), a layer of second-order retinal neurons (corresponding to the inner nuclear layer of the mature retina), a layer of photoreceptor (PR) cells, and an outer layer of RPE cells.

8. The method of claim 7, wherein each of the layers comprise differentiated cells characteristic of the cells within the corresponding layer of human retinal tissue.

9. The method of claim 7, wherein each of the layers comprise progenitor cells and wherein some or all or the progenitor cells differentiate into mature cells of the corresponding layer of human retinal tissue after administration.

10. The method of claim 7, wherein the layers comprise substantially fully differentiated cells.

11. The method of claim 1 or 2, wherein the hESC-derived retinal tissue further comprises a biocompatible scaffold to form a bioprosthetic retinal patch.

12. The method of claim 7, wherein the bioprosthetic retinal graft comprises between about 10,000 and 100,000 photoreceptor cells.

13. The method of claim 11, wherein several pieces of the hESC-derived retinal tissue are affixed to the biocompatible scaffold, such that a large bioprosthetic patch is formed.

14. The method of claim 6, wherein the hESC-derived retinal tissue graft or dissociated cells of the hESC derived retinal tissue graft are capable of delivering to a subject one or more of, neurotrophic factors, neurotrophic exosomes and mitogens.

15. The method of claim 14, wherein the neurotrophic factors and mitogens comprise one or more of, brain-derived neurotrophic factor (BDNF), glial-derived neurotrophic factor (GDNF), neurotrophin-34 (NT34), neurotrophin 4/5, Nerve Growth Factor -beta (βNGF), proNGF, PEDF, CNTF, pro-survival mitogen basic fibroblast growth factor (bFGF=FGF-2) and pro-survival members of the WNT family.

16. The method of claim 1 or 2, wherein administration of the hESC-derived retinal tissue graft results in preservation of retinal layer thickness for between about 1 to about 3 months where administered.

17. The method of claim 1 or 2, further comprising administration of immunosuppressive drugs.

18. The method of claim 1 or 2, further comprising administration of epinephrine before, during and/or after administering the retinal graft.

19. The method of claim 17, wherein the immunosuppressive drugs are administered before, during and/or after the administration.

20. The method of claim 1, wherein the method further comprises modulating the ocular pressure.

21. The method of claim 20, wherein the modulating the ocular pressure is before, during and/or after the administration of the retinal tissue.

22. The method of claim 1, wherein the tissue is administered with an ocular grafting tool.

23. The method of claim 1 or 2, wherein the hESC-derived retinal tissue is administered subretinally or epiretinally.

24. The method of claim 1 or 2, wherein administration of the hESC-derived retinal tissue graft results in tumor-free integration of the hESC-derived retinal tissue and retinal tissue of the subject.

25. The method of claim 24, wherein integration of retinal graft occurs between about 2 to 10 weeks after administration.

26. The method of claim 25, wherein integration comprises structural integration.

27. The method of claim 24, wherein integration comprises functional integration and occurs between about 1 to 6 months after administration.

28. The method of claim 1, wherein administering does not cause retinal inflammation.

29. The retinal tissue graft of claim 26, wherein after administering, the retinal tissue develops lamination.

30. The method of claim 1, wherein after administering, the retinal tissue neurons show signs of Na⁺, K⁺ and/or Ca⁺⁺ currents.

31. The method of claim 1, further comprising, demonstrating connectivity between the retinal tissue and existing tissue.

32. The method of claim 31, wherein the connection is demonstrated by one or more of:
WGA-HRP trans-synaptic tracer, histology, IHC or electrophysiology.

33. The method of claim 1, further comprising measuring a level of functional recovery.

34. The method of claim 33, wherein a level of functional recovery comprises a gain in the electrophysiological responses that is at least 10% of a baseline.

35. Retinal tissue graft for transplantation into an eye of a subject, comprising:
retinal pigmented epithelial (RPE) cells, retinal ganglion cells (RGCs), second-order retinal neurons, and photoreceptor (PR) cells, wherein the RPE, RGC and PR cells are configured to form a central core.

36. The retinal tissue graft of claim 35, wherein there are from between about 1,000 and 250,000 photoreceptors.

37. The retinal tissue graft of claim 35, wherein the second-order retinal neurons correspond to the inner nuclear layer of the mature retina.

38. The retinal tissue graft of claim 35, wherein the cells are arranged such that moving radially outward from the core, the retinal tissue comprises a layer of retinal ganglion cells (RGCs), a layer of second-order retinal neurons, a layer of photoreceptor (PR) cells, and an outer layer of RPE cells.

39. The retinal tissue graft of claim 35, wherein the graft comprises from between 1,000 to about 250,000 cells.

40. The retinal tissue graft of claim 35, wherein the graft is transplanted into the subretinal space or epiretinal space.

41. The retinal tissue graft of claim 40, wherein the graft is transplanted into the subretinal space or epiretinal space near the macula.

42. The retinal tissue graft of claim 35, wherein an increase in synaptogenesis coincides with increase in electric activity.

43. The retinal tissue graft of claim 35, wherein after transplantation neurons connect the graft to existing tissue.

44. The retinal tissue graft of claim 43, wherein the neurons are CALB2-positive.

45. The retinal tissue of claim 43, wherein connectivity is demonstrated by WGA-HRP trans-synaptic tracer.

46. The retinal tissue graft of claim 35, wherein after transplantation axons connect the graft to existing tissue.

47. The retinal tissue of claim 46, wherein the axons are CALB2-positive.

48. The retinal tissue graft of claim 35, wherein after transplantation, cells of the graft mature toward RGCs.

49. The retinal tissue graft of claim 35, wherein after transplantation the graft forms synapses with existing neurons.

50. The retinal tissue graft of claim 35, wherein after transplantation the graft and existing tissue form connections.

51. The retinal tissue of claim 50, wherein the connections form within one day to about 5 weeks after transplantation.

52. The retinal tissue graft of claim 35, wherein after transplantation the graft forms axons which cross the existing tissue ONL.

53. The retinal tissue graft of claim 35, wherein the graft produces paracrine factors.

54. The retinal tissue graft of claim 53, wherein the paracrine factors are produced prior and/or after to administration.

55. The retinal tissue graft of claim 35, wherein the graft produces neurotrophic factors.

56. The retinal tissue graft of claim 55, wherein the graft produces neurotrophic factors prior to or after administration.

57. The retinal tissue of claim 55, wherein the neurotrophic factors comprise one or more of, BDNS, GDNF, bNGF, NT4, bFGF, NT34, NT4/5, CNTF, PEDF, serpins, or WNT family members.

58. The retinal tissue graft of claim 35, wherein after transplantation, the level of functional recovery is measured as a gain in the electrophysiological responses.

59. The retinal tissue graft of claim 58, wherein the level of functional recovery is measured as a gain in the electrophysiological responses to at least 10% of a baseline.

60. The retinal tissue graft of claim 35, wherein after transplantation, axons of the graft penetrate and integrate into existing tissue.

61. The retinal tissue graft of claim 35, wherein the tissue is derived from human pluripotent stem cells.

62. The retinal tissue graft of claim 35, wherein the graft is useful for slowing the progression of retinal degenerative disease, slowing the progression of retinal degenerative disease after traumatic injury, slowing the progression of age related macular degeneration (AMD), slowing the progression of genetic retinal diseases, stabilizing retinal disease, preventing retinal degenerative disease, preventing retinal degenerative disease after traumatic injury, improving vision or visual perception, preventing AMD, restoring retinal pigment epithelium (RPE), photoreceptor cells (PRCs) and retinal ganglion cells (RGCs) lost from disease, injury or genetic abnormalities, increasing RPE, PRCs and RCGs or treating RPE, PRCs and RCG defects, in a subject.

63. The retinal tissue graft of claim 35, wherein the graft is capable of tumor-free survival for at least about 6 to 24 months, with lamination and development of PR and RPE layers, including elongating PR outer segments, synaptogenesis, electrophysiological activity and connectivity with recipient retinal cells after implantation into a recipient's ocular space.

64. The retinal tissue graft of claim 35, wherein the graft is capable of extending and integrating axons into a recipient's outer nuclear layer (ONL), into the inner nuclear layer (INL) and into the ganglion cell layer (GCL) after 5 weeks after the graft is implanted into the ocular space of the recipient's eye.
